(19) United States
(12) Patent Application Publication
(10) Pub. No.: US 2022/0379066 A1
(43) Pub. Date: Dec. 1, 2022

(54) METHODS AND APPARATUS FOR CONTROL OF OXYGEN CONCENTRATOR

(71) Applicant: ResMed Asia Pte. Ltd., Singapore (SG)

(72) Inventors: Rex Dael NAVARRO, Singapore (SG); Yong Sern GWEE, Singapore (SG)

(73) Assignee: ResMed Asia Pte. Ltd., Singapore (SG)

(21) Appl. No.: 17/770,886

(22) PCT Filed: Nov. 9, 2020
(86) PCT No.: PCT/SG2020/050644

§ 371 Date: Apr. 21, 2022

Related U.S. Application Data
(60) Provisional application No. 62/932,125, filed on Nov. 7, 2019.

(51) Int. Cl. A61M 16/10; A61M 16/20

(52) U.S. Cl. CPC A61M 16/101; A61M 16/202; A61M 16/1055; A61M 2016/003

(57) ABSTRACT

Methods and apparatus provide controlled operations in an oxygen concentrator (100) such as by adjusting valve opening time to regulate amount of oxygen enriched air released to a user. The apparatus may generate, with a sensor configured to sense pressure at a location associated with accumulation of enriched air produced by the concentrator, a signal representing measured pressure of the accumulated enriched air. The apparatus may generate, with a sensor, a signal indicative of respiration of a user of the concentrator. The apparatus may include a controller configured to receive the measured pressure and respiration signals. The controller may control, responsive to the respiration indication and according to a target duration, actuation of a valve adapted to release a bolus of accumulated oxygen enriched air. The controller may dynamically determine the target duration during the release of the bolus according to a function of a value of the measured pressure.

### CROSS-REFERENCE TO RELATED APPLICATIONS

The present disclosure claims priority from U.S. Provisional Patent Application Ser. No. 62/932,125, filed on Nov. 7, 2019, the entire disclosure of which is hereby incorporated by reference.

### FIELD OF THE TECHNOLOGY

The present technology relates generally to methods and apparatus for treating respiratory disorders, such as those involving gas adsorption or controlled pressure and/or vacuum swing adsorption. Such methodologies may be implemented in an oxygen concentrator using one or more sieve beds. In some examples, the technology more specifically concerns such methods and apparatus for a portable oxygen concentrator having a pulsed oxygen delivery or demand mode such as to regulate quantity of delivered gas, such as oxygen enriched air, or a desired bolus size.

### BACKGROUND

### The Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Examples of respiratory disorders include respiratory failure, Obesity Hyperventilation Syndrome (OHS), Chronic Obstructive Pulmonary Disease (COPD), Neuromuscular Disease (NMD) and Chest wall disorders.

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

### Therapies

Various respiratory therapies have been used to treat one or more of the above respiratory disorders.

### Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube. In some forms, the comfort and effectiveness of these therapies may be improved.

### Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched air. In one example, High Flow therapy (HFT) is the provision of a continuous, heated, humidified flow of air to an entrance to the airway through an unsealed or open patient interface at a “treatment flow rate” that is held approximately constant throughout the respiratory cycle. The treatment flow rate is nominally set to exceed the patient's peak inspiratory flow rate. HFT has been used to treat respiratory failure, COPD, and other respiratory disorders. One mechanism of action is that the high flow rate of air at the airway entrance improves ventilation efficiency by flushing, or washing out, expired CO₂ from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### Respiratory Therapy Systems

These respiratory therapies may be provided by a respiratory therapy system or device. Such systems and devices may also be used to screen, diagnose, or monitor a condition without treating it.

A respiratory therapy system may comprise an oxygen source, an air circuit, and a patient interface.

### Oxygen Source

Experts in this field have recognized that exercise for respiratory failure patients provides long term benefits that slow the progression of the disease, improve quality of life and extend patient longevity. Most stationary forms of exercise like tread mills and stationary bicycles, however, are too strenuous for these patients. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen. In order to make these devices small for mobility, the various systems necessary for the production of oxygen enriched air are condensed. POCs seek to utilize their produced oxygen as efficiently as possible, in order to minimise weight, size, and power consumption. This may be achieved by delivering the oxygen as series of pulses or “boluses”, each bolus timed to coincide with the onset of inhalation. Such a mode of operation may be implemented with a conserver. The therapy mode is known as pulsed oxygen delivery (POD) or demand mode, in contrast with traditional continuous flow delivery more suited to stationary oxygen concentrators.

Oxygen concentrators may implement processes such as vacuum swing adsorption (VSA), pressure swing adsorption (PSA), or vacuum pressure swing adsorption (VPSA). For example, oxygen concentrators, e.g., POCs, may work based on depressurization (e.g., vacuum operation) and/or pressurization (e.g., compressor operation) in a swing adsorption process (e.g., Vacuum Swing Adsorption VSA, Pressure Swing Adsorption PSA or Vacuum Pressure Swing Adsorption VPSA, each of which are referred to herein as a “swing adsorption process”). For example, an oxygen concentrator may control a process of pressure swing adsorption (PSA). Pressure swing adsorption involves using a compressor to increase gas pressure inside a canister that contains particles of a gas separation adsorbent that attracts nitrogen more strongly than it does oxygen. Such a canister filled with adsorbent is referred to as a sieve bed. Ambient air usually includes approximately 78% nitrogen and 21% oxygen with the balance comprised of argon, carbon dioxide, water vapor and other trace gases. If a feed gas mixture such as air, for example, is passed under pressure through a sieve bed, part or all of the nitrogen will be adsorbed by the sieve bed, and the gas coming out of the vessel will be enriched in oxygen. When the sieve bed reaches the end of its capacity to adsorb nitrogen, it can be regenerated by reducing the pressure, thereby releasing the adsorbed nitrogen. It is then ready for another “PSA cycle” of producing oxygen enriched air. By alternating canisters in a two-canister system, one canister can be concentrating oxygen (the so-called “adsorption phase”) while the other canister is being purged (the “purge phase”). This alternation results in a continuous separation of the oxygen from the nitrogen. In this manner, oxygen can be continuously concentrated out of the air for a variety of uses include providing LTOT to users.

Vacuum swing adsorption (VSA) provides an alternative gas separation technique. VSA typically draws the gas through the separation process of the sieve beds using a vacuum such as a compressor configured to create a vacuum with the sieve beds. Vacuum Pressure Swing Adsorption (VPSA) may be understood to be a hybrid system using a combined vacuum and pressurization technique. For example, a VPSA system may pressurize the sieve beds for the separation process and also apply a vacuum for purging of the beds.

### Air Circuit

An air circuit is a conduit or a tube constructed and arranged to allow, in use, a flow of breathable gas to travel between two components of a respiratory therapy system such as the oxygen source and the patient interface. In some cases, there may be separate limbs of the air circuit for inhalation and exhalation. In other cases, a single limb air circuit is used for both inhalation and exhalation.

### Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal LTOT, the patient interface is configured to insufflate the nares but specifically to avoid a complete seal. One example of such a patient interface is a nasal cannula.

An oxygen concentrator may control oxygen enriched air release in a pulsed or demand mode. This may be achieved by delivering the oxygen as a series of pulses, where each pulse or “bolus” may be timed to coincide with inspiration. Such a mode is typically controlled by actuating a pneumatic valve that releases oxygen enriched air for a fixed time. The fixed time is calibrated to be associated with a desired or target bolus size, e.g. a target bolus volume. However, such a fixed-time bolus release process does not always achieve the target bolus volume. For example, system characteristics such as compressor variability as well as the adsorption process (e.g., the PSA cycle, sieve bed condition, air filter condition etc.) can affect the delivered bolus size, leading to variability in the delivered bolus size that exceeds acceptable bounds of performance.

A need therefore exists for methods and apparatus for bolus release that regulate the delivered bolus size more closely to the target volume.

### SUMMARY OF THE TECHNOLOGY

Examples of the present technology may provide methods and apparatus for controlled operations of an oxygen concentrator, such as a portable oxygen concentrator. In particular, the technology provides methods and apparatus for a portable oxygen concentrator having a control mode to regulate amount of released oxygen enriched air, such as by controlling the release of a bolus to achieve a target bolus size (e.g., volume) more reliably. In some forms, the methods and apparatus dynamically control the timing of actuation of a supply valve that releases the bolus. The dynamic control adapts the timing to changes in the measured pressure of an accumulator from which the oxygen enriched air is delivered. The dynamic control is partly based on a model of bolus volume in terms of supply valve actuation timing and accumulator pressure. In some implementations, the dynamic control may be modified to achieve a desired target bolus size based a measure of temperature of the oxygen enriched air to be delivered.

Some versions of the present technology may include a method of operating an oxygen concentrator. The method may include generating, with a sensor configured to sense pressure at a location associated with accumulation of oxygen enriched air produced by the oxygen concentrator, a signal representing measured pressure of the accumulated oxygen enriched air. The method may include generating, with a sensor, a signal indicative of respiration of a user of the oxygen concentrator. The method may include with a controller configured to receive the signal representing measured pressure and the signal indicative of a respiration of the user, controlling, responsive to the signal indicative of respiration and according to a target duration, actuation of a valve adapted to release a bolus of the accumulated oxygen enriched air. The method may include the controller dynamically determining the target duration during the release of the bolus according to a function of a value of the measured pressure.

In some versions, the controller may control actuation of the valve by (a) opening the valve to initiate release of the bolus at a first time associated with a detection of an inspiration characteristic in the signal indicative of respiration of the user, and (b) closing the valve when elapsed time from the first time meets or exceeds the target duration. The controller may close the valve when the elapsed time from the first time meets or exceeds a maximum time. The controller may refrain from closing the valve until the elapsed time from the first time meets or exceeds a minimum time. Optionally the value of the measured pressure may be a calculated average. The calculated average may be an average pressure during the bolus release. In some versions, the controller may (a) repeatedly update the average pressure and the target duration during the release of the bolus, and may (b) repeatedly compare the elapsed time and the updated target duration during the release of the bolus.

In some versions, the function may include a target bolus size. The controller may calculate the target bolus size as a function of a detected respiration rate of the user and a flow rate associated with a flow setting of the oxygen concentrator. The method may include generating, with a sensor, a signal indicative of a temperature of the accumulated oxygen enriched air. The controller may adjust the target bolus size dependent on the signal indicative of the temperature of the accumulated oxygen enriched air.

The function may include a plurality of empirical constants of a modelled surface derived from pressure values and valve opening times of a calibration process. The modelled surface may be bilinear. In some versions, the function may include:

$$TargetDuration = \frac{( TargetBolusSize - a * P - d )}{( b * P + c )} ,$$

where TargetDuration may be the target duration, TargetBolusSize may be a target bolus size, P may be the value of the measured pressure; and a, b, c and d may be the empirical constants. The empirical constants may include a selected set of empirical constants associated with a flow rate setting of the oxygen concentrator. The selected set may be chosen from a plurality of discrete sets of empirical constants that are respectively associated with a plurality of discrete flow rate settings of the oxygen concentrator.

In some versions, the controller may include: an idle state, a start state, a bolus estimation state, and a stop state. The controller may transition from the idle state to the start state upon detecting an inspiration characteristic in the signal indicative of respiration of the user. The controller, in the start state, may generate a signal to open the valve, and initialize a valve timer. The controller, in the start state, may calculate an average pressure value with samples taken from the signal representing measured pressure in the start state. The controller may transition to the bolus estimation state from the start state when the valve timer exceeds a minimum time. The controller, in the bolus estimation state, may repeatedly calculate a target duration with the average pressure value. The controller, in the bolus estimation state, may repeatedly calculate the average pressure value with samples taken from the signal representing measured pressure in the bolus estimation state. The controller, in the bolus estimation state, may repeatedly compare the target duration with the valve timer. The controller may transition to the stop state when (a) the valve timer meets or exceeds the target duration, or (b) when the valve timer meets or exceeds a maximum time. The controller, in the stop state, may stop generating the signal to open the valve.

Some versions of the present technology may include an oxygen concentrator. The oxygen concentrator may include one or more sieve beds containing a gas separation adsorbent. The oxygen concentrator may include a compression system, including a motor operated compressor, configured to feed a feed gas into the one or more sieve beds. The oxygen concentrator may include an accumulator configured to receive oxygen enriched air from the one or more sieve beds. The oxygen concentrator may include a respiration sensor configured to generate a signal indicative of respiration of a user of the oxygen concentrator. The oxygen concentrator may include a pressure sensor configured to generate a signal representing a measure of pressure of the oxygen enriched air in the accumulator. The oxygen concentrator may include a valve adapted to release a bolus of the oxygen enriched air from the accumulator. The oxygen concentrator may include a memory. The oxygen concentrator may include a controller, which may include one or more processors. The one or more processors may be configured by program instructions stored in the memory to execute the method of operating the oxygen concentrator according to any one or more of the method(s) described herein.

Some versions of the present technology may include a computer-readable medium having encoded thereon computer-readable instructions that when executed by a controller of an oxygen concentrator cause the controller to perform the method of operating the oxygen concentrator of according to any one or more of the method(s) described herein.

Some versions of the present technology may include an oxygen concentrator. The oxygen concentrator may include one or more sieve beds containing a gas separation adsorbent. The oxygen concentrator may include a compression system, such as including a motor operated compressor, configured to feed a feed gas into the one or more sieve beds. The oxygen concentrator may include an accumulator to receive oxygen enriched air from the one or more sieve beds. The oxygen concentrator may include a pressure sensor configured to generate a signal representing measured pressure of the oxygen enriched air in the accumulator. The oxygen concentrator may include a respiration sensor configured to generate a signal indicative of respiration of a user of the oxygen concentrator. The oxygen concentrator may include a valve adapted to release a bolus of the oxygen enriched air from the accumulator. The oxygen concentrator may include a controller coupled with the pressure sensor, the respiration sensor and the valve. The controller may be configured to receive the signal representing measured pressure. The controller may be configured to receive the signal indicative of respiration. The controller may be configured to control, responsive to the signal indicative of respiration and according to a target duration, actuation of the valve to release the bolus of the oxygen enriched air. The controller may be configured to dynamically determine the target duration during the release of the bolus according to a function of a value of the measured pressure.

In some versions, the controller may be configured to control actuation of the valve by (a) opening the valve to initiate release of the bolus at a first time associated with a detection of an inspiration characteristic in the signal indicative of respiration of the user, and (b) closing the valve when elapsed time from the first time meets or exceeds the target duration. The controller may be configured to close the valve when the elapsed time from the first time meets or exceeds a maximum time. The controller may be configured to refrain from closing the valve until the elapsed time from the first time meets or exceeds a minimum time. The value of the measured pressure may be a calculated average. The calculated average may be an average pressure during the release of the bolus. The controller may be configured to (a) repeatedly update the average pressure and the target duration during the bolus release, and (b) repeatedly compare the elapsed time with the updated target duration during the release of the bolus. The function may include a target bolus size. The controller may be configured to calculate the target bolus size as a function of a detected respiration rate of the user and a flow rate associated with a flow setting of the oxygen concentrator. The oxygen concentrator may further include a sensor configured to generate a signal indicative of a temperature of the oxygen enriched air in the accumulator. The controller may be configured to adjust the target bolus size dependent on the signal indicative of the temperature of the oxygen enriched air.

In some versions, the function may include a plurality of empirical constants of a modelled surface derived from pressure values and valve opening times of a calibration process. The modelled surface may be bilinear. The function may be determined as:

$$TargetDuration = \frac{( TargetBolusSize - a * P - d )}{( b * P + c )}$$

where: TargetDuration may be the target duration, TargetBolusSize may be a target bolus size, P may be the value of the measured pressure, and a, b, c and d are the empirical constants. The empirical constants may include a selected set of empirical constants associated with a flow rate setting of the oxygen concentrator. The controller may be configured to choose the selected set from a plurality of discrete sets of empirical constants that are respectively associated with a plurality of discrete flow rate settings of the oxygen concentrator.

In some versions, to regulate bolus release, the controller may be configured with: an idle state, a start state, a bolus estimation state and a stop state. The controller may be configured to transition from the idle state to the start state upon detection of an inspiration characteristic in the signal indicative of respiration of the user. The controller, in the start state, may be configured to generate a signal to open the valve, and initialize a valve timer. The controller, in the start state, may be configured to calculate an average pressure value with samples taken from the signal representing measured pressure in the start state. The controller may be configured to transition to the bolus estimation state from the start state when the valve timer exceeds a minimum time. The controller, in the bolus estimation state, may be configured to repeatedly calculate a target duration with the average pressure value. The controller, in the bolus estimation state, may be configured to repeatedly calculate the average pressure value with samples taken from the signal representing measured pressure in the bolus estimation state. The controller, in the bolus estimation state, may be configured to repeatedly compare the target duration with the valve timer. The controller may be configured to transition to the stop state when (a) the valve timer meets or exceeds the target duration, or (b) when the valve timer meets or exceeds a maximum time. The controller, in the stop state, may be configured to stop generating the signal to open the valve.

Some versions of the present technology may include apparatus. The apparatus may include bed means for containing a gas separation adsorbent. The apparatus may include means for feeding a feed gas into the bed means. The apparatus may include accumulation means for receiving oxygen enriched air from the bed means. The apparatus may include pressure sensing means for generating a signal representing measured pressure of the oxygen enriched air in the accumulation means. The apparatus may include respiration sensing means for generating a signal indicative of respiration of a user of the apparatus. The apparatus may include releasing means adapted to release a bolus of the oxygen enriched air from the accumulation means. The apparatus may include controlling means coupled with the pressure sensing means, the respiration sensing means and the releasing means. The controlling means may be for receiving the signal representing measured pressure. The controlling means may be for receiving the signal indicative of respiration. The controlling means may be for controlling, responsive to the signal indicative of respiration and according to a target duration, actuation of the releasing means to release the bolus of the accumulated oxygen enriched air. The controlling means may be for dynamically determining the target duration during the release of the bolus according to a function of a value of the measured pressure.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### BRIEF DESCRIPTION OF THE DRAWINGS

Advantages of the present technology will become apparent to those skilled in the art with the benefit of the following detailed description of implementations and upon reference to the accompanying drawings in which:

FIG. 1 depicts an oxygen concentrator in accordance with one form of the present technology.

FIG. 2 is a schematic diagram of the pneumatic system of the oxygen concentrator of FIG. 1.

FIG. 3 is a side view of the main components of the oxygen concentrator of FIG. 1.

FIG. 4 is a perspective side view of a compression system of the oxygen concentrator of FIG. 1.

FIG. 5 is a side view of a compression system that includes a heat exchange conduit.

FIG. 6 is a schematic diagram of example outlet components of the oxygen concentrator of FIG. 1.

FIG. 7 depicts an outlet conduit for the oxygen concentrator of FIG. 1.

FIG. 8 depicts an alternate outlet conduit for the oxygen concentrator of FIG. 1.

FIG. 9 is a perspective view of a disassembled canister system for the oxygen concentrator of FIG. 1.

FIG. 10 is an end view of the canister system of FIG. 9.

FIG. 11 is an assembled view of the canister system end depicted in FIG. 10.

FIG. 12 a view of an opposing end of the canister system of FIG. 9 to that depicted in FIGS. 10 and 11.

FIG. 13 is an assembled view of the canister system end depicted in FIG. 12.

FIG. 14 depicts an example control panel for the oxygen concentrator of FIG. 1.

FIG. 15 is a flow chart of an example methodology for controlling bolus release such as with a controller of the oxygen concentrator of FIG. 1.

FIG. 16 is a graph illustrating a calibration process for determining modelling coefficients useful for application with the bolus release control that may be implemented with the control methodology of FIG. 15.

FIG. 17 is an example state machine for operation in a controller of the oxygen concentrator of FIG. 1 utilizing the methodology of FIG. 15.

FIG. 18 is a graph illustrating bolus size over time for an oxygen concentrator operating without the methodology of FIG. 15.

FIG. 19 is a graph illustrating bolus size over time for an oxygen concentrator operating with the methodology of FIG. 15 such as according to examples of the technology described in more detail herein.

### DETAILED DESCRIPTION OF THE IMPLEMENTATIONS

An example adsorption device of the present technology involving an oxygen concentrator may be considered in relation to the examples of the figures. The examples of the present technology may be implemented with any of the following structures and operations.

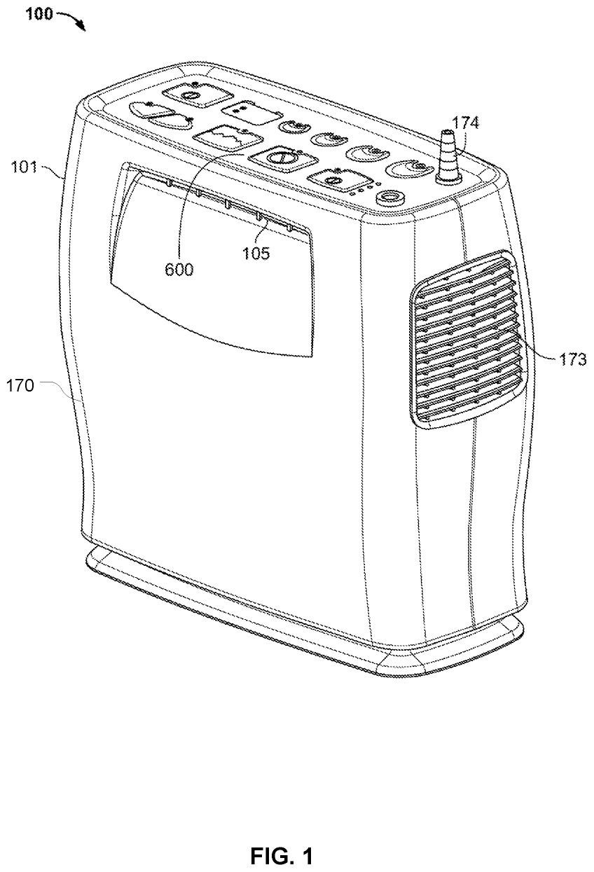

FIGS. 1 to 14 illustrate an implementation of an oxygen concentrator 100. Oxygen concentrator 100 may concentrate oxygen within an air stream to provide oxygen enriched air to a user. Oxygen concentrator 100 may be a portable oxygen concentrator. For example, oxygen concentrator 100 may have a weight and size that allows the oxygen concentrator to be carried by hand and/or in a carrying case. In one implementation, oxygen concentrator 100 has a weight of less than about 20 pounds, less than about 15 pounds, less than about 10 pounds, or less than about 5 pounds. In an implementation, oxygen concentrator 100 has a volume of less than about 1000 cubic inches, less than about 750 cubic inches, less than about 500 cubic inches, less than about 250 cubic inches, or less than about 200 cubic inches.

As described herein, oxygen concentrator 100 uses a pressure swing adsorption (PSA) process (which is cyclic) to produce oxygen enriched air. However, in other implementations, oxygen concentrator 100 may be modified such that it uses a cyclic vacuum swing adsorption (VSA) process or a cyclic vacuum pressure swing adsorption (VPSA) process to produce oxygen enriched air.

### Outer Housing

FIG. 1 depicts an implementation of an outer housing 170 of an oxygen concentrator 100. In some implementations, outer housing 170 may be comprised of a light-weight plastic. Outer housing includes compression system inlets 105, cooling system passive inlet 101 and outlet 173 at each end of outer housing 170, outlet port 174, and control panel 600. Inlet 101 and outlet 173 allow cooling air to enter the housing, flow through the housing, and exit the interior of housing 170 to aid in cooling of the oxygen concentrator 100. Compression system inlets 105 allow air to enter the compression system. Outlet port 174 is used to attach a conduit to provide oxygen enriched air produced by the oxygen concentrator 100 to a user.

### Pneumatic System

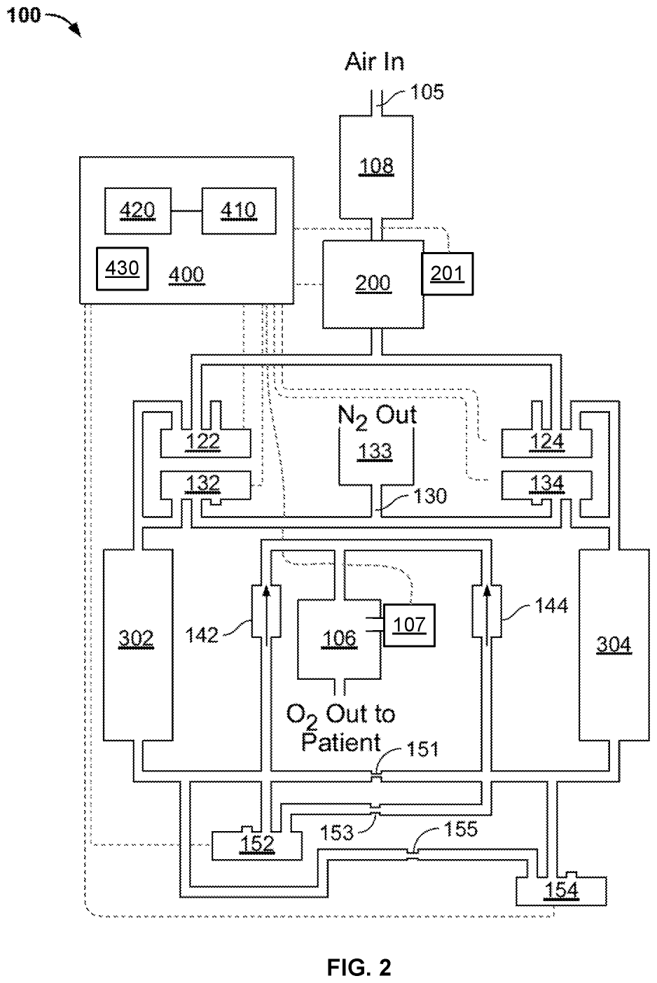

FIG. 2 is a schematic diagram of a pneumatic system of an oxygen concentrator such as the oxygen concentrator 100, according to an implementation. The pneumatic system may concentrate oxygen within an air stream to provide oxygen enriched air to an outlet system (described below).

Oxygen enriched air may be produced from ambient air by pressurising ambient air in canisters 302 and 304, which contain a gas separation adsorbent and are therefore referred to as sieve beds. Gas separation adsorbents useful in an oxygen concentrator are capable of separating at least nitrogen from an air stream to produce oxygen enriched air. Examples of gas separation adsorbents include molecular sieves that are capable of separating nitrogen from an air stream. Examples of adsorbents that may be used in an oxygen concentrator include, but are not limited to, zeolites (natural) or synthetic crystalline aluminosilicates that separate nitrogen from an air stream under elevated pressure. Examples of synthetic crystalline aluminosilicates that may be used include, but are not limited to: OXYSIV adsorbents available from UOP LLC, Des Plaines, Iowa; SYLOBEAD adsorbents available from W. R. Grace & Co, Columbia, Md.; SILIPORITE adsorbents available from CECA S.A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 2, air may enter the oxygen concentrator through air inlet 105. Air may be drawn into air inlet 105 by compression system 200. Compression system 200 may draw in air from the surroundings of the oxygen concentrator and compress the air, forcing the compressed air into one or both canisters 302 and 304. In an implementation, an inlet muffler 108 may be coupled to air inlet 105 to reduce sound produced by air being pulled into the oxygen concentrator by compression system 200. In an implementation, inlet muffler 108 may be a moisture and sound absorbing muffler. For example, a water absorbent material (such as a polymer water absorbent material or a zeolite material) may be used to both absorb water from the incoming air and to reduce the sound of the air passing into the air inlet 105.

Compression system 200 may include one or more compressors configured to compress air. Pressurized air, produced by compression system 200, may be forced into one or both of the canisters 302 and 304. In some implementations, the ambient air may be pressurized in the canisters to a pressure approximately in a range of 13-20 pounds per square inch gauge pressure (psig). Other pressures may also be used, depending on the type of gas separation adsorbent disposed in the canisters.

Coupled to each canister 302/304 are inlet valves 122/124 and outlet valves 132/134. As shown in FIG. 2, inlet valve 122 is coupled to canister 302 and inlet valve 124 is coupled to canister 304. Outlet valve 132 is coupled to canister 302 and outlet valve 134 is coupled to canister 304. Inlet valves 122/124 are used to control the passage of air from compression system 200 to the respective canisters. Outlet valves 132/134 are used to release gas from the respective canisters during a venting process. In some implementations, inlet valves 122/124 and outlet valves 132/134 may be silicon plunger solenoid valves. Other types of valves, however, may be used. Plunger valves offer advantages over other kinds of valves by being quiet and having low slippage.

In some implementations, a two-step valve actuation voltage may be used to control inlet valves 122/124 and outlet valves 132/134. For example, a high voltage (e.g., 24 V) may be applied to an inlet valve to open the inlet valve. The voltage may then be reduced (e.g., to 7 V) to keep the inlet valve open. Using less voltage to keep a valve open may use less power (Power=Voltage*Current). This reduction in voltage minimizes heat buildup and power consumption to extend run time from the battery. When the power is cut off to the valve, it closes by spring action. In some implementations, the voltage may be applied as a function of time that is not necessarily a stepped response (e.g., a curved downward voltage between an initial 24 V and a final 7 V).

In an implementation, pressurized air is sent into one of canisters 302 or 304 while the other canister is being vented. For example, during use, inlet valve 122 is opened while inlet valve 124 is closed. Pressurized air from compression system 200 is forced into canister 302, while being inhibited from entering canister 304 by inlet valve 124. In an implementation, a controller 400 is electrically coupled to valves 122, 124, 132, and 134. Controller 400 includes one or more processors 410 operable to execute program instructions stored in memory 420. The program instructions configure the controller to perform various predefined methods that are used to operate the oxygen concentrator, such as the methods described in more detail herein. The program instructions may include program instructions for operating inlet valves 122 and 124 out of phase with each other, i.e., when one of inlet valves 122 or 124 is opened, the other valve is closed. During pressurization of canister 302, outlet valve 132 is closed and outlet valve 134 is opened. Similar to the inlet valves, outlet valves 132 and 134 are operated out of phase with each other. In some implementations, the voltages and the durations of the voltages used to open the input and output valves may be controlled by controller 400.

The controller 400 may include a transceiver 430 that may communicate with external devices to transmit data collected by the processor 410 or receive instructions from an external computing device for the processor 410.

Check valves 142 and 144 are coupled to canisters 302 and 304, respectively. Check valves 142 and 144 may be one-way valves that are passively operated by the pressure differentials that occur as the canisters are pressurized and vented, or may be active valves. Check valves 142 and 144 are coupled to the canisters to allow oxygen enriched air produced during pressurization of each canister to flow out of the canister, and to inhibit back flow of oxygen enriched air or any other gases into the canister. In this manner, check valves 142 and 144 act as one-way valves allowing oxygen enriched air to exit the respective canisters during pressurization.

The term “check valve”, as used herein, refers to a valve that allows flow of a fluid (gas or liquid) in one direction and inhibits back flow of the fluid. Examples of check valves that are suitable for use include, but are not limited to: a ball check valve; a diaphragm check valve; a butterfly check valve; a swing check valve; a duckbill valve; an umbrella valve; and a lift check valve. Under pressure, nitrogen molecules in the pressurized ambient air are adsorbed by the gas separation adsorbent in the pressurized canister. As the pressure increases, more nitrogen is adsorbed until the gas in the canister is enriched in oxygen. The nonadsorbed gas molecules (mainly oxygen) flow out of the pressurized canister when the pressure reaches a point sufficient to overcome the resistance of the check valve coupled to the canister. In one implementation, the pressure drop of the check valve in the forward direction is less than 1 psig. The break pressure in the reverse direction is greater than 100 psig. It should be understood, however, that modification of one or more components would alter the operating parameters of these valves. If the forward flow pressure is increased, there is, generally, a reduction in oxygen enriched air production. If the break pressure for reverse flow is reduced or set too low, there is, generally, a reduction in oxygen enriched air pressure.

In an exemplary implementation, canister 302 is pressurized by compressed air produced in compression system 200 and passed into canister 302. During pressurization of canister 302 inlet valve 122 is open, outlet valve 132 is closed, inlet valve 124 is closed and outlet valve 134 is open. Outlet valve 134 is opened when outlet valve 132 is closed to allow substantially simultaneous venting of canister 304 to atmosphere while canister 302 is being pressurized. Canister 302 is pressurized until the pressure in canister is sufficient to open check valve 142. Oxygen enriched air produced in canister 302 exits through check valve and, in one implementation, is collected in accumulator 106.

After some time, the gas separation adsorbent will become saturated with nitrogen and will be unable to separate significant amounts of nitrogen from incoming air. This point is usually reached after a predetermined time of oxygen enriched air production. In the implementation described above, when the gas separation adsorbent in canister 302 reaches this saturation point, the inflow of compressed air is stopped and canister 302 is vented to remove nitrogen. During venting, inlet valve 122 is closed, and outlet valve 132 is opened. While canister 302 is being vented, canister 304 is pressurized to produce oxygen enriched air in the same manner described above. Pressurization of canister 304 is achieved by closing outlet valve 134 and opening inlet valve 124. The oxygen enriched air exits canister 304 through check valve 144.

During venting of canister 302, outlet valve 132 is opened allowing pressurized gas (mainly nitrogen) to exit the canister to atmosphere through concentrator outlet 130. In an implementation, the vented gases may be directed through muffler 133 to reduce the noise produced by releasing the pressurized gas from the canister. As gas is released from canister 302, the pressure in the canister 302 drops, allowing the nitrogen to become desorbed from the gas separation adsorbent. The released nitrogen exits the canister through outlet 130, resetting the canister to a state that allows renewed separation of nitrogen from an air stream. Muffler 133 may include open cell foam (or another material) to muffle the sound of the gas leaving the oxygen concentrator. In some implementations, the combined muffling components/techniques for the input of air and the output of oxygen enriched air may provide for oxygen concentrator operation at a sound level below 50 decibels.

During venting of the canisters, it is advantageous that at least a majority of the nitrogen is removed. In an implementation, at least about 50%, at least about 60%, at least about 70%, at least about 80%, at least about 90%, at least about 95%, at least about 98%, or substantially all of the nitrogen in a canister is removed before the canister is re-used to separate nitrogen from air. In some implementations, a canister may be further purged of nitrogen using an oxygen enriched air stream that is introduced into the canister from the other canister.

In an exemplary implementation, a portion of the oxygen enriched air may be transferred from canister 302 to canister 304 when canister 304 is being vented of nitrogen. Transfer of oxygen enriched air from canister 302 to 304 during venting of canister 304, helps to further purge nitrogen (and other gases) from the canister. In an implementation, oxygen enriched air may travel through flow restrictors 151, 153, and 155 between the two canisters. Flow restrictor 151 may be a trickle flow restrictor. Flow restrictor 151, for example, may be a 0.009D flow restrictor (e.g., the flow restrictor has a radius 0.009″ which is less than the diameter of the tube it is inside). Flow restrictors 153 and 155 may be 0.013D flow restrictors. Other flow restrictor types and sizes are also contemplated and may be used depending on the specific configuration and tubing used to couple the canisters. In some implementations, the flow restrictors may be press fit flow restrictors that restrict air flow by introducing a narrower diameter in their respective tube. In some implementations, the press fit flow restrictors may be made of sapphire, metal or plastic (other materials are also contemplated).

Flow of oxygen enriched air between the canisters is also controlled by use of valve 152 and valve 154. Valves 152 and 154 may be opened for a short duration during the venting process (and may be closed otherwise) to prevent excessive oxygen loss out of the purging canister. Other durations are also contemplated. In an exemplary implementation, canister 302 is being vented and it is desirable to purge canister 302 by passing a portion of the oxygen enriched air being produced in canister 304 into canister 302. A portion of oxygen enriched air, upon pressurization of canister 304, will pass through flow restrictor 151 into canister 302 during venting of canister 302. Additional oxygen enriched air is passed into canister 302, from canister 304, through valve 154 and flow restrictor 155. Valve 152 may remain closed during the transfer process, or may be opened if additional oxygen enriched air is needed. The selection of appropriate flow restrictors 151 and 155, coupled with controlled opening of valve 154 allows a controlled amount of oxygen enriched air to be sent from canister 304 to canister 302. In an implementation, the controlled amount of oxygen enriched air is an amount sufficient to purge canister 302 and minimize the loss of oxygen enriched air through venting valve 132 of canister 302. While this implementation describes venting of canister 302, it should be understood that the same process can be used to vent canister 304 using flow restrictor 151, valve 152 and flow restrictor 153.

The pair of equalization/vent valves 152/154 work with flow restrictors 153 and 155 to optimize the gas flow balance between the two canisters. This may allow for better flow control for venting one of the canisters with oxygen enriched air from the other of the canisters. It may also provide better flow direction between the two canisters. It has been found that, while flow valves 152/154 may be operated as bi-directional valves, the flow rate through such valves varies depending on the direction of fluid flowing through the valve. For example, oxygen enriched air flowing from canister 304 toward canister 302 has a flow rate faster through valve 152 than the flow rate of oxygen enriched air flowing from canister 302 toward canister 304 through valve 152. If a single valve was to be used, eventually either too much or too little oxygen enriched air would be sent between the canisters and the canisters would, over time, begin to produce different amounts of oxygen enriched air. Use of opposing valves and flow restrictors on parallel air pathways may equalize the flow pattern of the oxygen enriched air between the two canisters. Equalising the flow may allow for a steady amount of oxygen enriched air to be available to the user over multiple cycles and also may allow a predictable volume of oxygen enriched air to purge the other of the canisters. In some implementations, the air pathway may not have restrictors but may instead have a valve with a built-in resistance or the air pathway itself may have a narrow radius to provide resistance.

At times, oxygen concentrator may be shut down for a period of time. When an oxygen concentrator is shut down, the temperature inside the canisters may drop as a result of the loss of adiabatic heat from the compression system. As the temperature drops, the volume occupied by the gases inside the canisters will drop. Cooling of the canisters may lead to a negative pressure in the canisters. Valves (e.g., valves 122, 124, 132, and 134) leading to and from the canisters are dynamically sealed rather than hermetically sealed. Thus, outside air may enter the canisters after shutdown to accommodate the pressure differential. When outside air enters the canisters, moisture from the outside air may be adsorbed by the gas separation adsorbent. Adsorption of water inside the canisters may lead to gradual degradation of the gas separation adsorbents, steadily reducing ability of the gas separation adsorbents to produce oxygen enriched air.

In an implementation, outside air may be inhibited from entering canisters after the oxygen concentrator is shut down by pressurising both canisters prior to shutdown. By storing the canisters under a positive pressure, the valves may be forced into a hermetically closed position by the internal pressure of the air in the canisters. In an implementation, the pressure in the canisters, at shutdown, should be at least greater than ambient pressure. As used herein the term “ambient pressure” refers to the pressure of the surroundings in which the oxygen concentrator is located (e.g. the pressure inside a room, outside, in a plane, etc.). In an implementation, the pressure in the canisters, at shutdown, is at least greater than standard atmospheric pressure (i.e., greater than 760 mmHg (Torr), 1 atm, 101,325 Pa). In an implementation, the pressure in the canisters, at shutdown, is at least about 1.1 times greater than ambient pressure; is at least about 1.5 times greater than ambient pressure; or is at least about 2 times greater than ambient pressure.

In an implementation, pressurization of the canisters may be achieved by directing pressurized air into each canister from the compression system and closing all valves to trap the pressurized air in the canisters. In an exemplary implementation, when a shutdown sequence is initiated, inlet valves 122 and 124 are opened and outlet valves 132 and 134 are closed. Because inlet valves 122 and 124 are joined together by a common conduit, both canisters 302 and 304 may become pressurized as air and/or oxygen enriched air from one canister may be transferred to the other canister. This situation may occur when the pathway between the compression system and the two inlet valves allows such transfer. Because the oxygen concentrator operates in an alternating pressurize/venting mode, at least one of the canisters should be in a pressurized state at any given time. In an alternate implementation, the pressure may be increased in each canister by operation of compression system 200. When inlet valves 122 and 124 are opened, pressure between canisters 302 and 304 will equalize, however, the equalized pressure in either canister may not be sufficient to inhibit air from entering the canisters during shutdown. In order to ensure that air is inhibited from entering the canisters, compression system 200 may be operated for a time sufficient to increase the pressure inside both canisters to a level at least greater than ambient pressure. Regardless of the method of pressurization of the canisters, once the canisters are pressurized, inlet valves 122 and 124 are closed, trapping the pressurized air inside the canisters, which inhibits air from entering the canisters during the shutdown period.

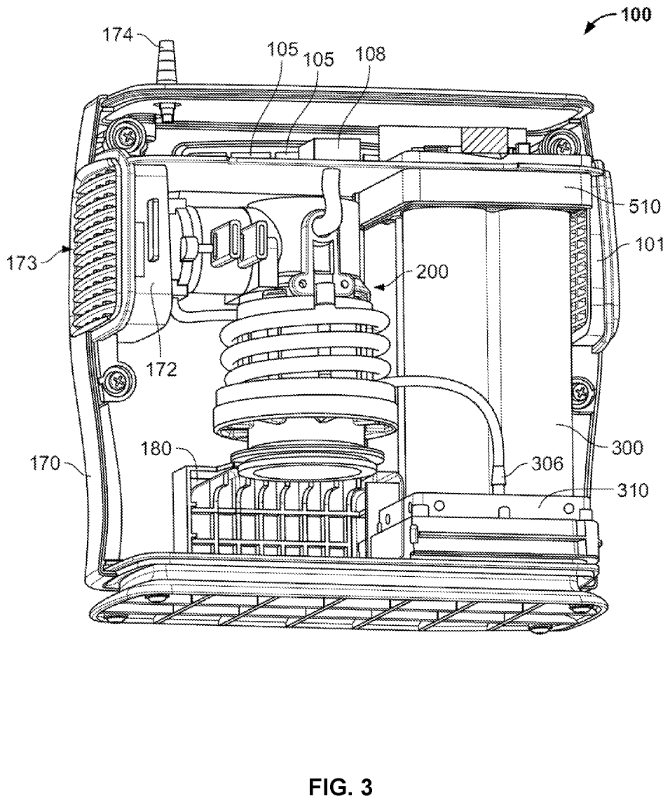

Referring to FIG. 3, an implementation of an oxygen concentrator 100 is depicted. Oxygen concentrator 100 includes a compression system 200, a canister system 300, and a power supply 180 disposed within an outer housing 170. Inlets 101 are located in outer housing 170 to allow air from the environment to enter oxygen concentrator 100. Inlets 101 may allow air to flow into the compartment to assist with cooling of the components in the compartment. Power supply 180 provides a source of power for the oxygen concentrator 100. Compression system 200 draws air in through the inlet 105 and muffler 108. Muffler 108 may reduce noise of air being drawn in by the compression system and also may include a desiccant material to remove water from the incoming air. Oxygen concentrator 100 may further include fan 172 used to vent air and other gases from the oxygen concentrator via outlet 173.

### Compression System

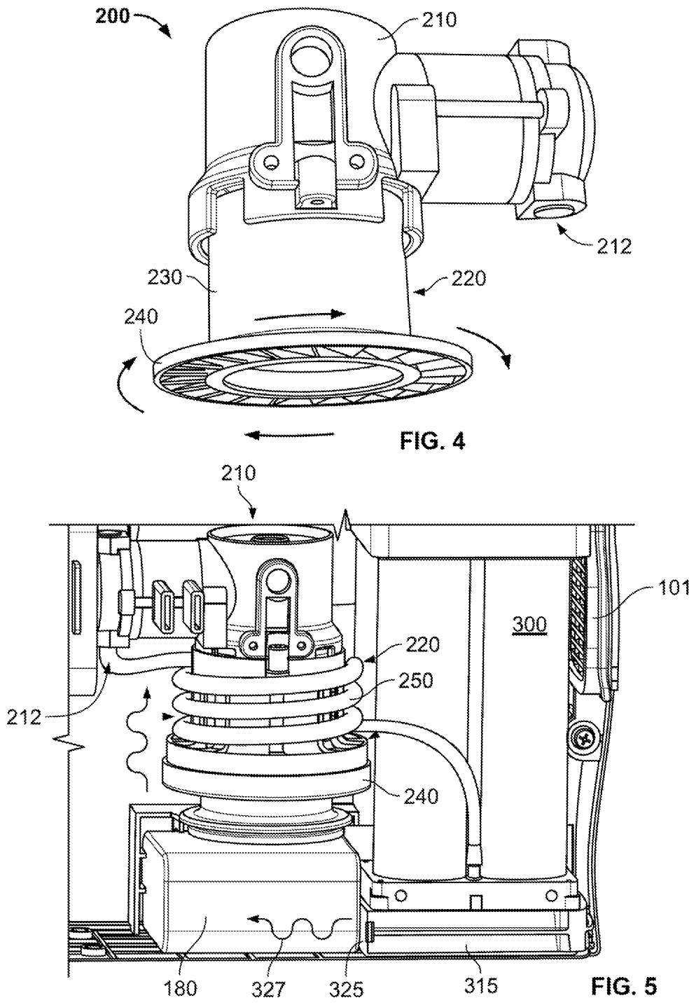

In some implementations, compression system 200 includes one or more compressors. In another implementation, compression system 200 includes a single compressor, coupled to all of the canisters of canister system 300. Turning to FIGS. 4 and 5, a compression system 200 is depicted that includes compressor 210 and motor 220. Motor 220 is coupled to compressor 210 and provides an operating force to the compressor to operate the compression mechanism. For example, motor 220 may be a motor providing a rotating component that causes cyclical motion of a component of the compressor that compresses air. When compressor 210 is a piston type compressor, motor 220 provides an operating force which causes the piston of compressor 210 to be reciprocated. Reciprocation of the piston causes compressed air to be produced by compressor 210. The pressure of the compressed air is, in part, estimated by the speed that the compressor is operated at, (e.g., how fast the piston is reciprocated). Motor 220, therefore, may be a variable speed motor that is operable at various speeds to dynamically control the pressure of air produced by compressor 210.

In one implementation, compressor 210 includes a single head wobble type compressor having a piston. Other types of compressors may be used such as diaphragm compressors and other types of piston compressors. Motor 220 may be a DC or AC motor and provides the operating power to the compressing component of compressor 210. Motor 220, in an implementation, may be a brushless DC motor. Motor 220 may be a variable speed motor configured to operate the compressing component of compressor 210 at variable speeds. Motor 220 may be coupled to controller 400, as depicted in FIG. 2, which sends operating signals to the motor to control the operation of the motor. For example, controller 400 may send signals to motor 220 to: turn the motor on, turn motor the off, and set the operating speed of the motor. Thus, as illustrated in FIG. 2, the compression system may include a speed sensor 201. The speed sensor may be a motor speed transducer used to determine a rotational velocity of the motor 220 and/or other reciprocating operation of the compression system 200. For example, a motor speed signal from the motor speed transducer may be provided to the controller 400. The speed sensor or motor speed transducer may, for example, be a Hall effect sensor. The controller 400 may operate the compression system via the motor 220 based on the speed signal and/or any other sensor signal of the oxygen concentrator, such as a pressure sensor (e.g., accumulator pressure sensor 107). Thus, as illustrated in FIG. 2, the controller 400 receives sensor signals, such as a speed signal from the speed sensor 201 and accumulator pressure signal from the accumulator pressure sensor 107. With such signal(s), the controller may implement one or more control loops (e.g., feedback control) for operation of the compression system based on sensor signals such as accumulator pressure and/or motor speed as described in more detail herein.

Compression system 200 inherently creates substantial heat. Heat is caused by the consumption of power by motor 220 and the conversion of power into mechanical motion. Compressor 210 generates heat due to the increased resistance to movement of the compressor components by the air being compressed. Heat is also inherently generated due to adiabatic compression of the air by compressor 210. Thus, the continual pressurization of air produces heat in the enclosure. Additionally, power supply 180 may produce heat as power is supplied to compression system 200. Furthermore, users of the oxygen concentrator may operate the device in unconditioned environments (e.g., outdoors) at potentially higher ambient temperatures than indoors, thus the incoming air will already be in a heated state.

Heat produced inside oxygen concentrator 100 can be problematic. Lithium ion batteries are generally employed as a power source for oxygen concentrators due to their long life and light weight. Lithium ion battery packs, however, are dangerous at elevated temperatures and safety controls are employed in oxygen concentrator 100 to shutdown the system if dangerously high power supply temperatures are detected. Additionally, as the internal temperature of oxygen concentrator 100 increases, the amount of oxygen generated by the concentrator may decrease. This is due, in part, to the decreasing amount of oxygen in a given volume of air at higher temperatures. If the amount of produced oxygen drops below a predetermined amount, the oxygen concentrator 100 may automatically shut down.

Because of the compact nature of oxygen concentrators, dissipation of heat can be difficult. Solutions typically involve the use of one or more fans to create a flow of cooling air through the enclosure. Such solutions, however, require additional power from the power supply and thus shorten the portable usage time of the oxygen concentrator. In an implementation, a passive cooling system may be used that takes advantage of the mechanical power produced by motor 220. Referring to FIGS. 4 and 5, compression system 200 includes motor 220 having an external rotating armature 230. Specifically, armature 230 of motor 220 (e.g. a DC motor) is wrapped around the stationary field that is driving the armature. Since motor 220 is a large contributor of heat to the overall system it is helpful to pull heat off the motor and sweep it out of the enclosure. With the external high speed rotation, the relative velocity of the major component of the motor and the air in which it exists is very high. The surface area of the armature is larger if externally mounted than if it is internally mounted. Since the rate of heat exchange is proportional to the surface area and the square of the velocity, using a larger surface area armature mounted externally increases the ability of heat to be dissipated from motor 220. The gain in cooling efficiency by mounting the armature externally, allows the elimination of one or more cooling fans, thus reducing the weight and power consumption while maintaining the interior of the oxygen concentrator within the appropriate temperature range. Additionally, the rotation of the externally mounted armature creates movement of air proximate to the motor to create additional cooling.

Moreover, an external rotating armature may help the efficiency of the motor, allowing less heat to be generated. A motor having an external armature operates similar to the way a flywheel works in an internal combustion engine. When the motor is driving the compressor, the resistance to rotation is low at low pressures. When the pressure of the compressed air is higher, the resistance to rotation of the motor is higher. As a result, the motor does not maintain consistent ideal rotational stability, but instead surges and slows down depending on the pressure demands of the compressor. This tendency of the motor to surge and then slow down is inefficient and therefore generates heat. Use of an external armature adds greater angular momentum to the motor which helps to compensate for the variable resistance experienced by the motor. Since the motor does not have to work as hard, the heat produced by the motor may be reduced.

In an implementation, cooling efficiency may be further increased by coupling an air transfer device 240 to external rotating armature 230. In an implementation, air transfer device 240 is coupled to the external armature 230 such that rotation of the external armature causes the air transfer device to create an air flow that passes over at least a portion of the motor. In an implementation, air transfer device includes one or more fan blades coupled to the armature. In an implementation, a plurality of fan blades may be arranged in an annular ring such that the air transfer device acts as an impeller that is rotated by movement of the external rotating armature. As depicted in FIGS. 4 and 5, air transfer device 240 may be mounted to an outer surface of the external armature 230, in alignment with the motor. The mounting of the air transfer device to the armature allows air flow to be directed toward the main portion of the external rotating armature, providing a cooling effect during use. In an implementation, the air transfer device directs air flow such that a majority of the external rotating armature is in the air flow path.

Further, referring to FIGS. 4 and 5, air pressurized by compressor 210 exits compressor 210 at compressor outlet 212. A compressor outlet conduit 250 is coupled to compressor outlet 212 to transfer the compressed air to canister system 300. As noted previously, compression of air causes an increase in the temperature of the air. This increase in temperature can be detrimental to the efficiency of the oxygen concentrator. In order to reduce the temperature of the pressurized air, compressor outlet conduit 250 is placed in the air flow path produced by air transfer device 240. At least a portion of compressor outlet conduit 250 may be positioned proximate to motor 220. Thus, air flow, created by air transfer device, may contact both motor 220 and compressor outlet conduit 250. In one implementation, a majority of compressor outlet conduit 250 is positioned proximate to motor 220. In an implementation, the compressor outlet conduit 250 is coiled around motor 220, as depicted in FIG. 5.

In an implementation, the compressor outlet conduit 250 is composed of a heat exchange metal. Heat exchange metals include, but are not limited to, aluminum, carbon steel, stainless steel, titanium, copper, copper-nickel alloys or other alloys formed from combinations of these metals. Thus, compressor outlet conduit 250 can act as a heat exchanger to remove heat that is inherently caused by compression of the air. By removing heat from the compressed air, the number of molecules in a given volume at a given pressure is increased. As a result, the amount of oxygen that can be generated by each canister during each pressure swing cycle may be increased.

The heat dissipation mechanisms described herein are either passive or make use of elements required for the oxygen concentrator 100. Thus, for example, dissipation of heat may be increased without using systems that require additional power. By not requiring additional power, the run-time of the battery packs may be increased and the size and weight of the oxygen concentrator may be minimized. Likewise, use of an additional box fan or cooling unit may be eliminated Eliminating such additional features reduces the weight and power consumption of the oxygen concentrator.

As discussed above, adiabatic compression of air causes the air temperature to increase. During venting of a canister in canister system 300, the pressure of the gas being released from the canisters decreases. The adiabatic decompression of the gas in the canister causes the temperature of the gas to drop as it is vented. In an implementation, the cooled vented gases 327 from canister system 300 are directed toward power supply 180 and toward compression system 200. In an implementation, base 315 of canister system 300 receives the vented gases from the canisters. The vented gases 327 are directed through base 315 toward outlet 325 of the base and toward power supply 180. The vented gases, as noted, are cooled due to decompression of the gases and therefore passively provide cooling to the power supply. When the compression system is operated, the air transfer device will gather the cooled vented gases and direct the gases toward the motor of compression system 200. Fan 172 may also assist in directing the vented gas across compression system 200 and out of the housing 170. In this manner, additional cooling may be obtained without requiring any further power requirements from the battery.

### Canister System

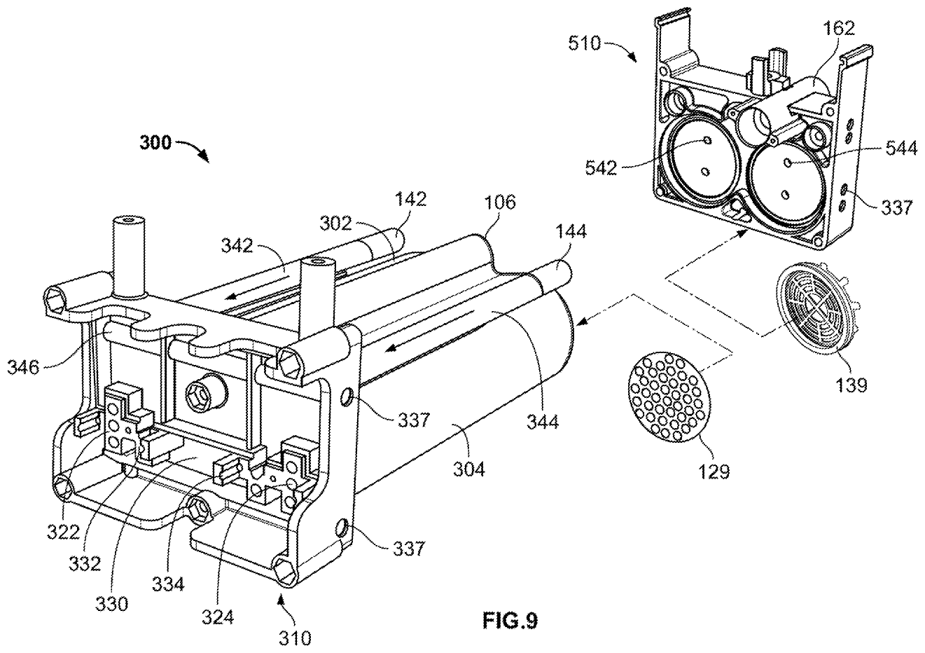

Oxygen concentrator 100 may include at least two canisters, each canister including a gas separation adsorbent. The canisters of oxygen concentrator 100 may be disposed formed from a molded housing. In an implementation, canister system 300 includes two housing components 310 and 510, as depicted in FIG. 9. In various implementations, the housing components 310 and 510 of the oxygen concentrator 100 may form a two-part molded plastic frame that defines two canisters 302 and 304 and accumulator 106. The housing components 310 and 510 may be formed separately and then coupled together. In some implementations, housing components 310 and 510 may be injection molded or compression molded. Housing components 310 and 510 may be made from a thermoplastic polymer such as polycarbonate, methylene carbide, polystyrene, acrylonitrile butadiene styrene (ABS), polypropylene, polyethylene, or polyvinyl chloride. In another implementation, housing components 310 and 510 may be made of a thermoset plastic or metal (such as stainless steel or a lightweight aluminum alloy). Lightweight materials may be used to reduce the weight of the oxygen concentrator 100. In some implementations, the two housings 310 and 510 may be fastened together using screws or bolts. Alternatively, housing components 310 and 510 may be solvent welded together.

As shown, valve seats 322, 324, 332, and 334 and air pathways of conduit 330 and 346 may be integrated into the housing component 310 to reduce the number of sealed connections needed throughout the air flow of the oxygen concentrator 100.

Air pathways/tubing between different sections in housing components 310 and 510 may take the form of molded conduits. Conduits in the form of molded channels for air pathways may occupy multiple planes in housing components 310 and 510. For example, the molded air conduits may be formed at different depths and at different x,y,z positions in housing components 310 and 510. In some implementations, a majority or substantially all of the conduits may be integrated into the housing components 310 and 510 to reduce potential leak points.

In some implementations, prior to coupling housing components 310 and 510 together, O-rings may be placed between various points of housing components 310 and 510 to ensure that the housing components are properly sealed. In some implementations, components may be integrated and/or coupled separately to housing components 310 and 510. For example, tubing, flow restrictors (e.g., press fit flow restrictors), oxygen sensors, gas separation adsorbents, check valves, plugs, processors, power supplies, etc. may be coupled to housing components 310 and 510 before and/or after the housing components are coupled together.

In some implementations, apertures 337 leading to the exterior of housing components 310 and 510 may be used to insert devices such as flow restrictors. Apertures may also be used for increased moldability. One or more of the apertures may be plugged after molding (e.g., with a plastic plug). In some implementations, flow restrictors may be inserted into passages prior to inserting plug to seal the passage. Press fit flow restrictors may have diameters that may allow a friction fit between the press fit flow restrictors and their respective apertures. In some implementations, an adhesive may be added to the exterior of the press fit flow restrictors to hold the press fit flow restrictors in place once inserted. In some implementations, the plugs may have a friction fit with their respective tubes (or may have an adhesive applied to their outer surface). The press fit flow restrictors and/or other components may be inserted and pressed into their respective apertures using a narrow tip tool or rod (e.g., with a diameter less than the diameter of the respective aperture). In some implementations, the press fit flow restrictors may be inserted into their respective tubes until they abut a feature in the tube to halt their insertion. For example, the feature may include a reduction in radius. Other features are also contemplated (e.g., a bump in the side of the tubing, threads, etc.). In some implementations, press fit flow restrictors may be molded into the housing components (e.g., as narrow tube segments).

In some implementations, spring baffle 139 may be placed into respective canister receiving portions of housing components 310 and 510 with the spring side of the baffle 139 facing the exit of the canister. Spring baffle 139 may apply force to gas separation adsorbent in the canister while also assisting in preventing gas separation adsorbent from entering the exit apertures. Use of a spring baffle 139 may keep the gas separation adsorbent compact while also allowing for expansion (e.g., thermal expansion). Keeping the gas separation adsorbent compact may prevent the gas separation adsorbent from breaking during movement of the oxygen concentrator 100.

In some implementations, filter 129 may be placed into respective canister receiving portions of housing components 310 and 510 facing the inlet of the respective canisters. The filter 129 removes particles from the feed gas stream entering the canisters.

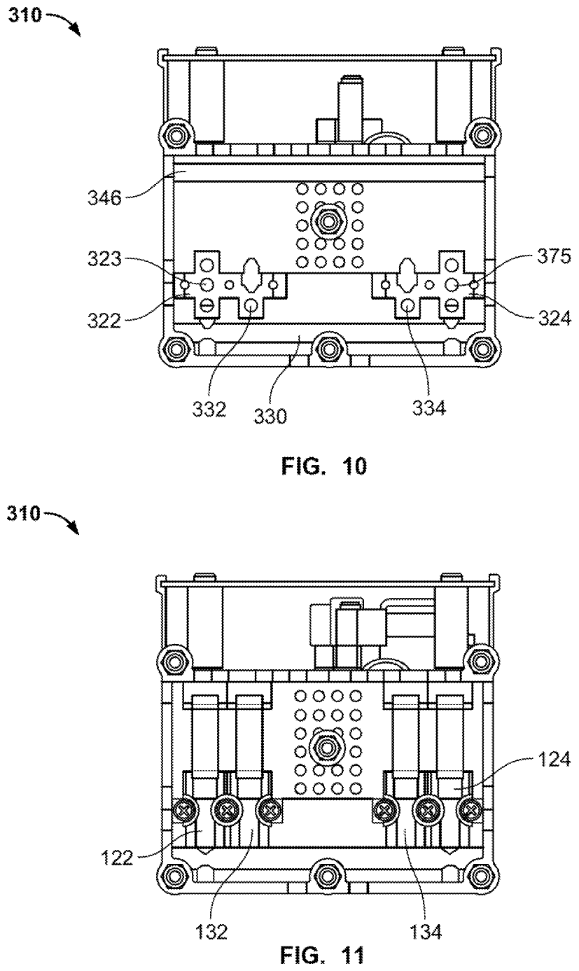

In some implementations, pressurized air from the compression system 200 may enter air inlet 306. Air inlet 306 is coupled to inlet conduit 330. Air enters housing component 310 through inlet 306 travels through conduit 330, and then to valve seats 322 and 324. FIG. 10 and FIG. 11 depict an end view of housing 310. FIG. 10 depicts an end view of housing 310 prior to fitting valves to housing 310. FIG. 11 depicts an end view of housing 310 with the valves fitted to the housing 310. Valve seats 322 and 324 are configured to receive inlet valves 122 and 124 respectively. Inlet valve 122 is coupled to canister 302 and inlet valve 124 is coupled to canister 304. Housing 310 also includes valve seats 332 and 334 configured to receive outlet valves 132 and 134 respectively. Outlet valve 132 is coupled to canister 302 and outlet valve 134 is coupled to canister 304. Inlet valves 122/124 are used to control the passage of air from conduit 330 to the respective canisters.

In an implementation, pressurized air is sent into one of canisters 302 or 304 while the other canister is being vented. For example, during use, inlet valve 122 is opened while inlet valve 124 is closed. Pressurized air from compression system 200 is forced into canister 302, while being inhibited from entering canister 304 by inlet valve 124. During pressurization of canister 302, outlet valve 132 is closed and outlet valve 134 is opened. Similar to the inlet valves, outlet valves 132 and 134 are operated out of phase with each other. Valve seat 322 includes an opening 323 that passes through housing 310 into canister 302. Similarly valve seat 324 includes an opening 375 that passes through housing 310 into canister 302. Air from conduit 330 passes through openings 323 or 375 if the respective valves 322 and 324 are open, and enters a canister.

Check valves 142 and 144 (See FIG. 9) are coupled to canisters 302 and 304, respectively. Check valves 142 and 144 are one way valves that may be passively operated by the pressure differentials that occur as the canisters are pressurized and vented. Oxygen enriched air produced in canisters 302 and 304 passes from the canisters into openings 542 and 544 of housing component 510. A passage (not shown) links openings 542 and 544 to conduits 342 and 344, respectively. Oxygen enriched air produced in canister 302 passes from the canister though opening 542 and into conduit 342 when the pressure in the canister is sufficient to open check valve 142. When check valve 142 is open, oxygen enriched air flows through conduit 342 toward the end of housing 310. Similarly, oxygen enriched air produced in canister 304 passes from the canister through opening 544 and into conduit 344 when the pressure in the canister is sufficient to open check valve 144. When check valve 144 is open, oxygen enriched air flows through conduit 344 toward the end of housing 310.

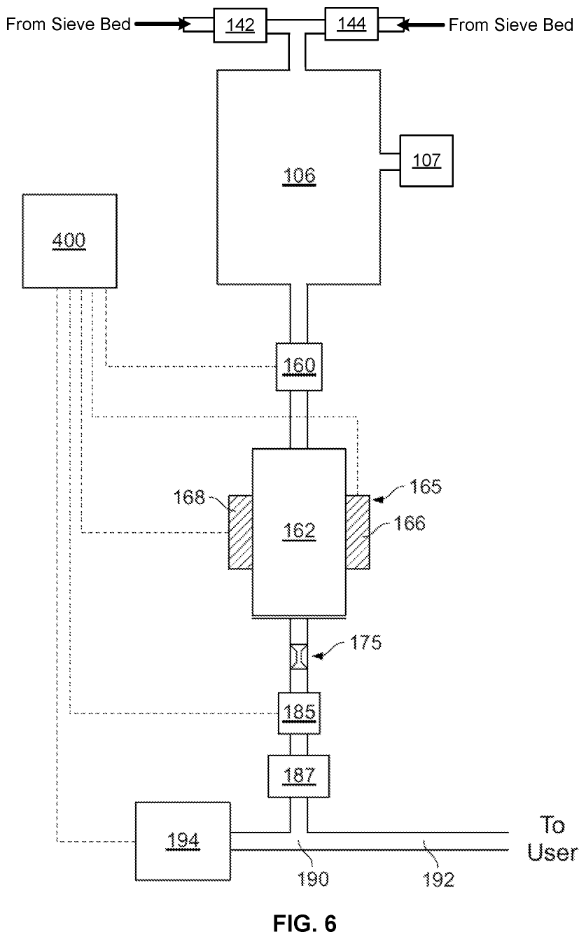

Oxygen enriched air from either canister travels through conduit 342 or 344 and enters conduit 346 formed in housing 310. Conduit 346 includes openings that couple the conduit to conduit 342, conduit 344 and accumulator 106. Thus, oxygen enriched air, produced in canister 302 or 304, travels to conduit 346 and passes into accumulator 106. As illustrated in FIG. 2, gas pressure within the accumulator 106 may be measured by a sensor, such as with an accumulator pressure sensor 107. (See also FIG. 6.) Thus, the accumulator pressure sensor provides a signal representing the pressure of the accumulated oxygen enriched air. An example of a suitable pressure transducer is a sensor from the HONEYWELL ASDX series. An alternative suitable pressure transducer is a sensor from the NPA Series from GENERAL ELECTRIC. In some versions, the pressure sensor may alternatively measure pressure of the gas outside of the accumulator 106, such as in an output path between the accumulator 106 and a valve (e.g., supply valve 160) that gates the release of the oxygen enriched air for delivery to a user in a bolus.

After some time, the gas separation adsorbent will become saturated with nitrogen and will be unable to separate significant amounts of nitrogen from incoming air. When the gas separation adsorbent in a canister reaches this saturation point, the inflow of compressed air is stopped and the canister is vented to remove nitrogen. Canister 302 is vented by closing inlet valve 122 and opening outlet valve 132. Outlet valve 132 releases the vented gas from canister 302 into the volume defined by the end of housing 310. Foam material may cover the end of housing 310 to reduce the sound made by release of gases from the canisters. Similarly, canister 304 is vented by closing inlet valve 124 and opening outlet valve 134. Outlet valve 134 releases the vented gas from canister 304 into the volume defined by the end of housing 310.

While canister 302 is being vented, canister 304 is pressurized to produce oxygen enriched air in the same manner described above. Pressurization of canister 304 is achieved by closing outlet valve 134 and opening inlet valve 124. The oxygen enriched air exits canister 304 through check valve 144.

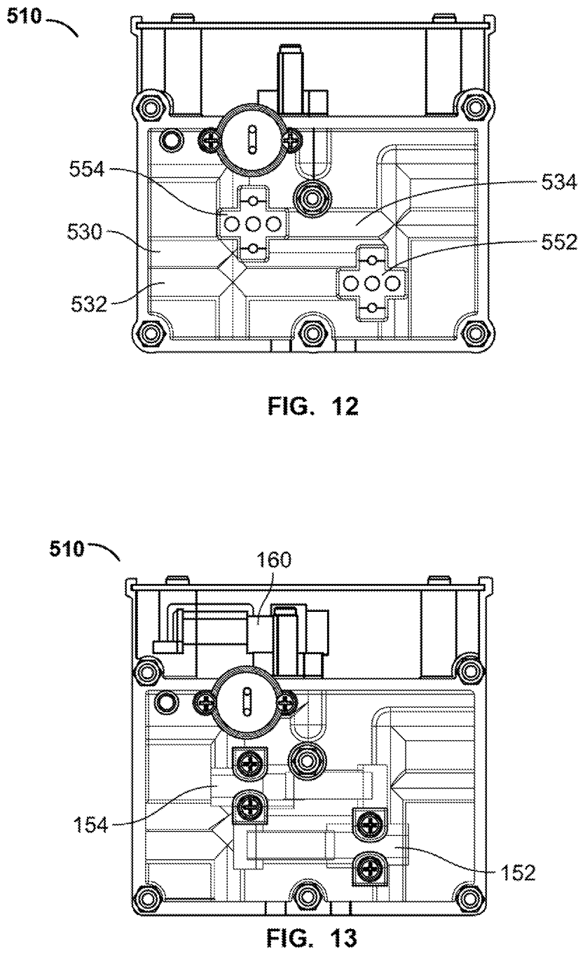

In an exemplary implementation, a portion of the oxygen enriched air may be transferred from canister 302 to canister 304 when canister 304 is being vented of nitrogen. Transfer of oxygen enriched air from canister 302 to canister 304, during venting of canister 304, helps to further purge nitrogen (and other gases) from the canister. Flow of oxygen enriched air between the canisters is controlled using flow restrictors and valves, as depicted in FIG. 2. Three conduits are formed in housing component 510 for use in transferring oxygen enriched air between canisters. As shown in FIG. 12, conduit 530 couples canister 302 to canister 304. Flow restrictor 151 (not shown) is disposed in conduit 530, between canister 302 and canister 304 to restrict flow of oxygen enriched air during use. Conduit 532 also couples canister 302 to 304. Conduit 532 is coupled to valve seat 552 which receives valve 152, as shown in FIG. 13. Flow restrictor 153 (not shown) is disposed in conduit 532, between canister 302 and 304. Conduit 534 also couples canister 302 to 304. Conduit 534 is coupled to valve seat 554 which receives valve 154, as shown in FIG. 13. Flow restrictor 155 (not shown) is disposed in conduit 534, between canister 302 and 304. The pair of equalization/vent valves 152/154 work with flow restrictors 153 and 155 to optimize the air flow balance between the two canisters.

Oxygen enriched air in accumulator 106 passes through supply valve 160 into expansion chamber 162 which is formed in housing component 510. An opening (not shown) in housing component 510 couples accumulator 106 to supply valve 160. In an implementation, expansion chamber 162 may include one or more devices configured to estimate an oxygen concentration of gas passing through the chamber.

### Outlet System

An outlet system, coupled to one or more of the canisters, includes one or more conduits for providing oxygen enriched air to a user. In an implementation, oxygen enriched air produced in either of canisters 302 and 304 is collected in accumulator 106 through check valves 142 and 144, respectively, as depicted schematically in FIG. 6. The oxygen enriched air leaving the canisters may be collected in an oxygen accumulator 106 prior to being provided to a user. In some implementations, a tube may be coupled to the accumulator 106 to provide the oxygen enriched air to the user. Oxygen enriched air may be provided to the user through an airway delivery device that transfers the oxygen enriched air to the user's mouth and/or nose. In an implementation, an outlet may include a tube that directs the oxygen toward a user's nose and/or mouth that may not be directly coupled to the user's nose.

Turning to FIG. 6, a schematic diagram of an implementation of an outlet system for an oxygen concentrator is shown. A supply valve 160 may be coupled to an outlet tube to control the release of the oxygen enriched air from accumulator 106 to the user. In an implementation, supply valve 160 is an electromagnetically actuated plunger valve. Supply valve 160 is actuated by controller 400 to control the delivery of oxygen enriched air to a user. Actuation of supply valve 160 is not timed or synchronized to the pressure swing adsorption process. Instead, actuation is synchronized to the user's breathing as described below. In some implementations, supply valve 160 may have continuously-valued actuation to establish a clinically effective amplitude profile for providing oxygen enriched air.

Oxygen enriched air in accumulator 106 passes through supply valve 160 into expansion chamber 162 as depicted in FIG. 6. In an implementation, expansion chamber 162 may include one or more devices configured to estimate an oxygen concentration of gas passing through the expansion chamber 162. Oxygen enriched air in expansion chamber 162 builds briefly, through release of gas from accumulator 106 by supply valve 160, and then is bled through a small orifice flow restrictor 175 to a flow rate sensor 185 and then to particulate filter 187. Flow restrictor 175 may be a 0.025 D flow restrictor. Other flow restrictor types and sizes may be used. In some implementations, the diameter of the air pathway in the housing may be restricted to create restricted gas flow. Optional flow rate sensor 185 may be any sensor configured to generate a signal representing the rate of gas flowing through the conduit. Particulate filter 187 may be used to filter bacteria, dust, granule particles, etc., prior to delivery of the oxygen enriched air to the user. The oxygen enriched air passes through filter 187 to connector 190 which sends the oxygen enriched air to the user via delivery conduit 192 and to pressure sensor 194.

The fluid dynamics of the outlet pathway, coupled with the programmed actuations of supply valve 160, may result in a bolus of oxygen being provided at the correct time and with an amplitude profile that assures rapid delivery into the user's lungs without excessive waste.

Expansion chamber 162 may include one or more oxygen sensors adapted to determine an oxygen concentration of gas passing through the chamber. In an implementation, the oxygen concentration of gas passing through expansion chamber 162 is estimated using an oxygen sensor 165. An oxygen sensor is a device configured to measure oxygen concentration in a gas. Examples of oxygen sensors include, but are not limited to, ultrasonic oxygen sensors, electrical oxygen sensors, chemical oxygen sensors, and optical oxygen sensors. In one implementation, oxygen sensor 165 is an ultrasonic oxygen sensor that includes an ultrasonic emitter 166 and an ultrasonic receiver 168. In some implementations, ultrasonic emitter 166 may include multiple ultrasonic emitters and ultrasonic receiver 168 may include multiple ultrasonic receivers. In implementations having multiple emitters/receivers, the multiple ultrasonic emitters and multiple ultrasonic receivers may be axially aligned (e.g., across the gas flow path which may be perpendicular to the axial alignment).

In use, an ultrasonic sound wave from emitter 166 may be directed through oxygen enriched air disposed in chamber 162 to receiver 168. The ultrasonic oxygen sensor 165 may be configured to detect the speed of sound through the oxygen enriched air to determine the composition of the oxygen enriched air. The speed of sound is different in nitrogen and oxygen, and in a mixture of the two gases, the speed of sound through the mixture may be an intermediate value proportional to the relative amounts of each gas in the mixture. In use, the sound at the receiver 168 is slightly out of phase with the sound sent from emitter 166. This phase shift is due to the relatively slow velocity of sound through a gas medium as compared with the relatively fast speed of the electronic pulse through wire. The phase shift, then, is proportional to the distance between the emitter and the receiver and inversely proportional to the speed of sound through the expansion chamber 162. The density of the gas in the chamber affects the speed of sound through the expansion chamber and the density is proportional to the ratio of oxygen to nitrogen in the expansion chamber. Therefore, the phase shift can be used to measure the concentration of oxygen in the expansion chamber. In this manner the relative concentration of oxygen in the accumulator may be estimated as a function of one or more properties of a detected sound wave traveling through the accumulator.

In some implementations, multiple emitters 166 and receivers 168 may be used. The readings from the emitters 166 and receivers 168 may be averaged to reduce errors that may be inherent in turbulent flow systems. In some implementations, the presence of other gases may also be detected by measuring the transit time and comparing the measured transit time to predetermined transit times for other gases and/or mixtures of gases.

The sensitivity of the ultrasonic sensor system may be increased by increasing the distance between the emitter 166 and receiver 168, for example to allow several sound wave cycles to occur between emitter 166 and the receiver 168. In some implementations, if at least two sound cycles are present, the influence of structural changes of the transducer may be reduced by measuring the phase shift relative to a fixed reference at two points in time. If the earlier phase shift is subtracted from the later phase shift, the shift caused by thermal expansion of expansion chamber 162 may be reduced or cancelled. The shift caused by a change of the distance between the emitter 166 and receiver 168 may be approximately the same at the measuring intervals, whereas a change owing to a change in oxygen concentration may be cumulative. In some implementations, the shift measured at a later time may be multiplied by the number of intervening cycles and compared to the shift between two adjacent cycles. Further details regarding sensing of oxygen in the expansion chamber may be found, for example, in U.S. Published Patent Application No. 2009-0065007, published Mar. 12, 2009, and entitled “Oxygen Concentrator Apparatus and Method”, which is incorporated herein by reference.

Flow rate sensor 185 may be used to determine the flow rate of gas flowing through the outlet system. Flow rate sensors that may be used include, but are not limited to: diaphragm/bellows flow meters; rotary flow meters (e.g. Hall effect flow meters); turbine flow meters; orifice flow meters; and ultrasonic flow meters. Flow rate sensor 185 may be coupled to controller 400. The rate of gas flowing through the outlet system may be an indication of the breathing volume of the user. Changes in the flow rate of gas flowing through the outlet system may also be used to determine a breathing rate of the user. Controller 400 may generate a control signal or trigger signal to control actuation of supply valve 160. Such control of actuation of the supply valve may be based on the breathing rate and/or breathing volume of the user, as estimated by flow rate sensor 185.

In some implementations, ultrasonic sensor 165 and, for example, flow rate sensor 185 may provide a measurement of an actual amount of oxygen being provided. For example, flow rate sensor 185 may measure a volume of gas (based on flow rate) provided and ultrasonic sensor 165 may provide the concentration of oxygen of the gas provided. These two measurements together may be used by controller 400 to determine an approximation of the actual amount of oxygen provided to the user.

Oxygen enriched air passes through flow rate sensor 185 to filter 187. Filter 187 removes bacteria, dust, granule particles, etc prior to providing the oxygen enriched air to the user. The filtered oxygen enriched air passes through filter 187 to connector 190. Connector 190 may be a “Y” connector coupling the outlet of filter 187 to pressure sensor 194 and delivery conduit 192. Pressure sensor 194 may be used to monitor the pressure of the gas passing through conduit 192 to the user. In some implementations, pressure sensor 194 is configured to generate a signal that is proportional to the amount of positive or negative pressure applied to a sensing surface. Changes in pressure, sensed by pressure sensor 194, may be used to determine a breathing rate of a user, as well as the onset of inhalation (also referred to as the trigger instant) as described below. Controller 400 may control actuation of supply valve 160 based on the breathing rate and/or onset of inhalation of the user. In an implementation, controller 400 may control actuation of supply valve 160 based on information provided by either or both of the flow rate sensor 185 and the pressure sensor 194.

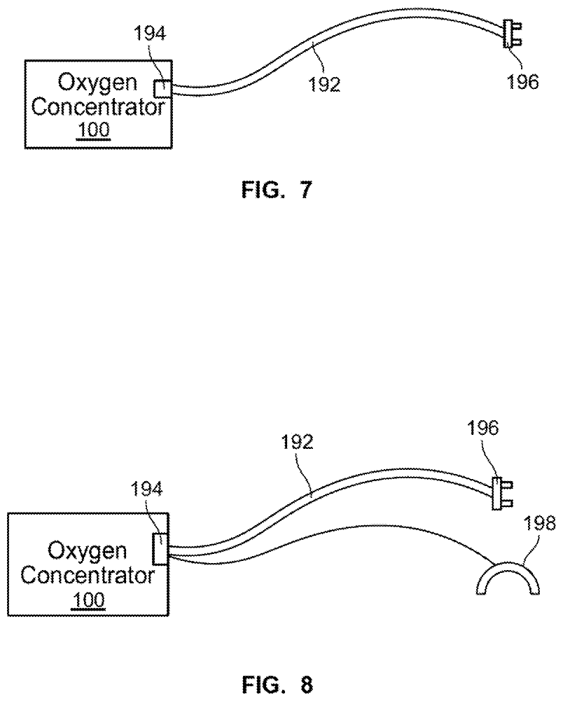

Oxygen enriched air may be provided to a user through conduit 192. In an implementation, conduit 192 may be a silicone tube. Conduit 192 may be coupled to a user using an airway delivery device 196, as depicted in FIGS. 7 and 8. Airway delivery device 196 may be any device capable of providing the oxygen enriched air to nasal cavities or oral cavities. Examples of airway delivery devices include, but are not limited to: nasal masks, nasal pillows, nasal prongs, nasal cannulas, and mouthpieces. A nasal cannula airway delivery device 196 is depicted in FIG. 7. Airway delivery device 196 is positioned proximate to a user's airway (e.g., proximate to the user's mouth and or nose) to allow delivery of the oxygen enriched air to the user while allowing the user to breathe air from the surroundings.

In an alternate implementation, a mouthpiece may be used to provide oxygen enriched air to the user. As shown in FIG. 8, a mouthpiece 198 may be coupled to oxygen concentrator 100. Mouthpiece 198 may be the only device used to provide oxygen enriched air to the user, or a mouthpiece may be used in combination with a nasal airway delivery device 196 (e.g., a nasal cannula). As depicted in FIG. 8, oxygen enriched air may be provided to a user through both a nasal airway delivery device 196 and a mouthpiece 198.

Mouthpiece 198 is removably positionable in a user's mouth. In one implementation, mouthpiece 198 is removably couplable to one or more teeth in a user's mouth. During use, oxygen enriched air is directed into the user's mouth via the mouthpiece. Mouthpiece 198 may be a night guard mouthpiece which is molded to conform to the user's teeth. Alternatively, mouthpiece may be a mandibular repositioning device. In an implementation, at least a majority of the mouthpiece is positioned in a user's mouth during use.

During use, oxygen enriched air may be directed to mouthpiece 198 when a change in pressure is detected proximate to the mouthpiece. In one implementation, mouthpiece 198 may be coupled to a pressure sensor 194. When a user inhales air through the user's mouth, pressure sensor 194 may detect a drop in pressure proximate to the mouthpiece. Controller 400 of oxygen concentrator 100 may control release of a bolus of oxygen enriched air to the user at the onset of inhalation.

During typical breathing of an individual, inhalation may occur through the nose, through the mouth or through both the nose and the mouth. Furthermore, breathing may change from one passageway to another depending on a variety of factors. For example, during more active activities, a user may switch from breathing through their nose to breathing through their mouth, or breathing through their mouth and nose. A system that relies on a single mode of delivery (either nasal or oral), may not function properly if breathing through the monitored pathway is stopped. For example, if a nasal cannula is used to provide oxygen enriched air to the user, an inhalation sensor (e.g., a pressure sensor or flow rate sensor) is coupled to the nasal cannula to determine the onset of inhalation. If the user stops breathing through their nose, and switches to breathing through their mouth, the oxygen concentrator 100 may not know when to provide the oxygen enriched air since there is no feedback from the nasal cannula. Under such circumstances, oxygen concentrator 100 may increase the flow rate and/or increase the frequency of providing oxygen enriched air until the inhalation sensor detects an inhalation by the user. If the user switches between breathing modes often, the default mode of providing oxygen enriched air may cause the oxygen concentrator 100 to work harder, limiting the portable usage time of the system.

In an implementation, a mouthpiece 198 is used in combination with a nasal airway delivery device 196 (e.g., a nasal cannula) to provide oxygen enriched air to a user, as depicted in FIG. 8. Both mouthpiece 198 and nasal airway delivery device 196 are coupled to an inhalation sensor. In one implementation, mouthpiece 198 and nasal airway delivery device 196 are coupled to the same inhalation sensor. In an alternate implementation, mouthpiece 198 and nasal airway delivery device 196 are coupled to different inhalation sensors. In either implementation, the inhalation sensor(s) may detect the onset of inhalation from either the mouth or the nose. Oxygen concentrator 100 may be configured to provide oxygen enriched air to the delivery device (i.e. mouthpiece 198 or nasal airway delivery device 196) proximate to which the onset of inhalation was detected. Alternatively, oxygen enriched air may be provided to both mouthpiece 198 and nasal airway delivery device 196 if onset of inhalation is detected proximate either delivery device. The use of a dual delivery system, such as depicted in FIG. 8 may be particularly useful for users when they are sleeping and may switch between nose breathing and mouth breathing without conscious effort.

### Controller System

Operation of oxygen concentrator 100 may be performed automatically using an internal controller 400 coupled to various components of the oxygen concentrator 100, as described herein. Controller 400 includes one or more processors 410 and internal memory 420, as depicted in FIG. 2. Methods used to operate and monitor oxygen concentrator 100 may be implemented by program instructions stored in internal memory 420 or an external memory medium coupled to controller 400, and executed by one or more processors 410. A memory medium may include any of various types of memory devices or storage devices. The term “memory medium” is intended to include an installation medium, e.g., a Compact Disc Read Only Memory (CD-ROM), floppy disks, or tape device; a computer system memory or random access memory such as Dynamic Random Access Memory (DRAM), Double Data Rate Random Access Memory (DDR RAM), Static Random Access Memory (SRAM), Extended Data Out Random Access Memory (EDO RAM), Random Access Memory (RAM), etc.; or a non-volatile memory such as a magnetic medium, e.g., a hard drive, or optical storage. The memory medium may comprise other types of memory as well, or combinations thereof. In addition, the memory medium may be located proximate to the controller 400 by which the programs are executed, or may be located in an external computing device that connects to the controller 400 over a network, such as the Internet. In the latter instance, the external computing device may provide program instructions to the controller 400 for execution. The term “memory medium” may include two or more memory media that may reside in different locations, e.g., in different computing devices that are connected over a network.

In some implementations, controller 400 includes processor 410 that includes, for example, one or more field programmable gate arrays (FPGAs), microcontrollers, etc. included on a circuit board disposed in oxygen concentrator 100. Processor 410 is configured to execute programming instructions stored in memory 420. In some implementations, programming instructions may be built into processor 410 such that a memory external to the processor 410 may not be separately accessed (i.e., the memory 420 may be internal to the processor 410).

Processor 410 may be coupled to various components of oxygen concentrator 100, including, but not limited to compression system 200, one or more of the valves used to control fluid flow through the system (e.g., valves 122, 124, 132, 134, 152, 154, 160), oxygen sensor 165, pressure sensor 194, flow rate sensor 185, temperature sensors (not shown), fan 172, and any other component that may be electrically controlled. In some implementations, a separate processor (and/or memory) may be coupled to one or more of the components.

Controller 400 is configured (e.g., programmed by program instructions) to operate oxygen concentrator 100 and is further configured to monitor the oxygen concentrator 100 such as for malfunction states or other process information. For example, in one implementation, controller 400 is programmed to trigger an alarm if the system is operating and no breathing is detected by the user for a predetermined amount of time. For example, if controller 400 does not detect a breath for a period of 75 seconds, an alarm LED may be lit and/or an audible alarm may be sounded. If the user has truly stopped breathing, for example, during a sleep apnea episode, the alarm may be sufficient to awaken the user, causing the user to resume breathing. The action of breathing may be sufficient for controller 400 to reset this alarm function. Alternatively, if the system is accidentally left on when delivery conduit 192 is removed from the user, the alarm may serve as a reminder for the user to turn oxygen concentrator 100 off.

Controller 400 is further coupled to oxygen sensor 165, and may be programmed for continuous or periodic monitoring of the oxygen concentration of the oxygen enriched air passing through expansion chamber 162. A minimum oxygen concentration threshold may be programmed into controller 400, such that the controller lights an LED visual alarm and/or an audible alarm to warn the user of the low concentration of oxygen.

Controller 400 is also coupled to internal power supply 180 and may be configured to monitor the level of charge of the internal power supply. A minimum voltage and/or current threshold may be programmed into controller 400, such that the controller lights an LED visual alarm and/or an audible alarm to warn the user of low power condition. The alarms may be activated intermittently and at an increasing frequency as the battery approaches zero usable charge.

Further functions that may be implemented with or by the controller 400 are described in detail in other sections of this disclosure.

For example, and as discussed in more detail herein including the detailed sections below, the controller of the POC may implement compressor control to regulate pressure in the system. Thus, the POC may be equipped with a pressure sensor such as in the accumulator downstream of the sieve beds. The controller 400 in the POC can control adjusting of the speed of the compressor using signals from the pressure sensor as well as a motor speed sensor such as in one or more modes. In this regard, the controller may implement dual control modes, designated a coarse pressure regulation mode and a fine pressure regulation mode. The coarse pressure regulation mode may be implemented for changing between the different flow rate settings (or “flow settings”) of the POC and for starting/initial activation. The fine pressure regulation mode may then take over upon completion of each operation of the coarse pressure regulation mode.

In the coarse pressure regulation mode, the motor speed is set/controlled to ramp up or down depending the prior state of operations. During the ramping, the controller uses the measurements from the pressure sensor to generate an estimated pressure upstream of the sensor, in the sieve beds. In some implementations, the estimated pressure is used in a test to terminate the ramp, e.g. when the estimated pressure reaches a predetermined pressure target, created at manufacturing time, that is associated with the selected flow rate setting of the POC. The pressure estimate may be calculated by performing a regression (e.g., linear) using data from the pressure sensor whereby the controller determines regression parameters (e.g., slope and intercept parameters of a line) from the sensor signal samples. The pressure estimate is calculated with the regression parameters and a known system response delay.

In the fine pressure regulation mode, the motor is set/controlled to maintain the pressure of the system using the signal from pressure sensor. Upon completion of the coarse pressure regulation mode, the motor speed ramping is stopped (i.e., the speed is maintained at a base speed) and any further changes to the base motor speed resulting from the coarse mode may be instead implemented with two controllers such as PID (proportional, integral, derivative) controllers. During the fine pressure regulation mode, the target pressure is compared with a qualified pressure estimate to generate a first error signal that is applied to the first controller (e.g. a PID controller) to produce, by summing the PID output of the PID controller with the base speed of the motor, a motor speed setting for control of the motor using a second controller (e.g. a PID controller). The qualified pressure estimate for the first PID controller is computed using regression. In this regard, samples from the pressure signal may be applied to a best fit algorithm (e.g., linear regression) to determine regression parameters (e.g., slope and intercept of a line) of the data from the pressure signal during an adsorption cycle. If the slope is positive, these parameters (slope and intercept rather than pressure samples from the pressure sensor) may then be applied with the particular time of the given adsorption phase of the pressure swing adsorption cycle to determine a peak value of the regression line from the linear regression. If the slope is negative, the intercept parameter may be taken as the peak value. The peak values from the regression information may be then applied to a running average buffer that maintains an average of the most recent peak values (e.g., six or more). The average peak value may then serve as the qualified pressure estimate for the controller. Versions of such processes are discussed in more detail in U.S. Provisional Patent Application No. 62/904,858 filed on 24 Sep. 2019 or Patent Cooperation Treaty Application No. PCT/AU2020/051015, filed on 24 Sep. 2020, the entire disclosures of which are incorporated herein by reference.

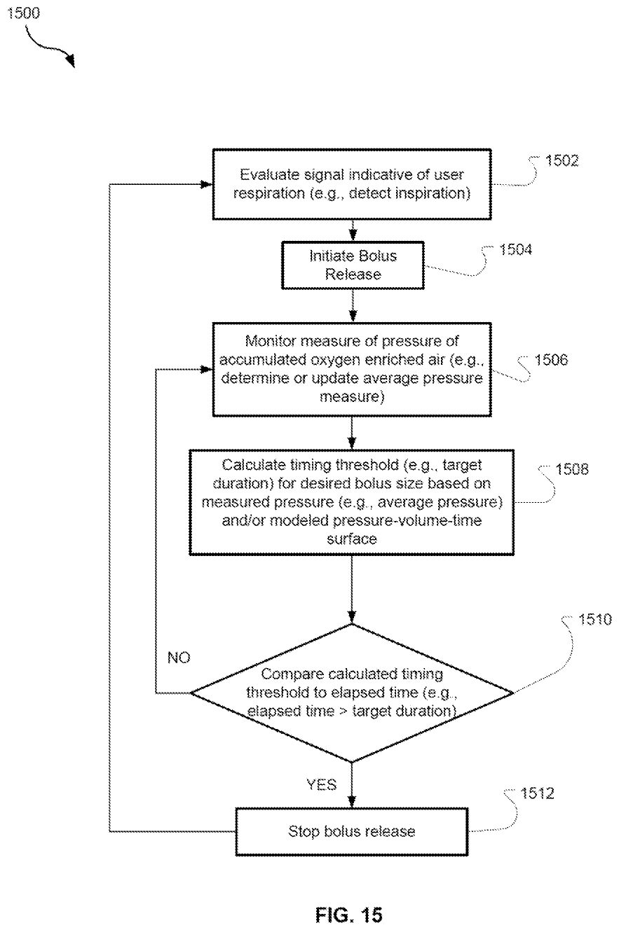

Additionally, as discussed in more detail herein, the controller of the POC may be configured to implement bolus control to regulate bolus size in the system, which may optionally be implemented without use of a flow rate sensor of the POC. For example, the POC may be equipped with a pressure sensor, such as in the accumulator downstream of the sieve beds, and regulate bolus size, generated by the POC, as a function of pressure. Such regulation of bolus size may be a function of pressure and valve timing. Examples of such control of operations are described in more detail below such as in relation to FIGS. 15, 16, 17 and 19.

### Control Panel

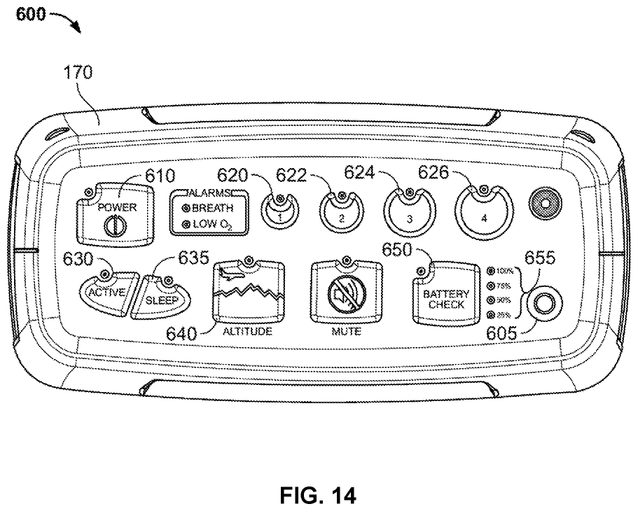

Control panel 600 serves as an interface between a user and controller 400 to allow the user to initiate predetermined operation modes of the oxygen concentrator 100 and to monitor the status of the system. FIG. 14 depicts an implementation of control panel 600. Charging input port 605, for charging the internal power supply 180, may be disposed in control panel 600.

In some implementations, control panel 600 may include buttons to activate various operation modes for the oxygen concentrator 100. For example, control panel may include power button 610, flow rate setting buttons 620 to 626, active mode button 630, sleep mode button 635, altitude button 640, and a battery check button 650. In some implementations, one or more of the buttons may have a respective LED that may illuminate when the respective button is pressed, and may power off when the respective button is pressed again. Power button 610 may power the system on or off. If the power button is activated to turn the system off, controller 400 may initiate a shutdown sequence to place the system in a shutdown state (e.g., a state in which both canisters are pressurized). Flow rate setting buttons 620, 622, 624, and 626 allow a flow rate of oxygen enriched air to be selected (e.g., 0.2 LPM by button 620, 0.4 LPM by button 622, 0.6 LPM by button 624, and 0.8 LPM by button 626). Altitude button 640 may be activated when a user is going to be in a location at a higher elevation than the oxygen concentrator 100 is regularly used by the user.

Battery check button 650 initiates a battery check routine in the oxygen concentrator 100 which results in a relative battery power remaining LED 655 being illuminated on control panel 600.

A user may have a low breathing rate or depth if relatively inactive (e.g., asleep, sitting, etc.) as estimated by comparing the detected breathing rate or depth to a threshold. The user may have a high breathing rate or depth if relatively active (e.g., walking, exercising, etc.). An active/sleep mode may be estimated automatically and/or the user may manually indicate active mode or sleep mode by pressing button 630 for active mode or button 635 for sleep mode.

### Methods of Operating the POC

The methods of operating and monitoring the POC 100 described below may be executed by the one or more processors, such as the one or more processors 410 of the controller 400, configured by program instructions, such as including, as previously described, the one or more functions and/or associated data corresponding thereto, stored in a memory such as the memory 420 of the POC 100. Alternatively, some or all of the steps of the described methods may be similarly executed by one or more processors of an external computing device to which the controller is connected via the transceiver 430. In this latter implementation, the processors 410 may be configured by program instructions stored in the memory 420 of the POC 100 to transmit to the external computing device the measurements and parameters necessary for the performance of those steps that are to be carried out at the external computing device.

The main use of an oxygen concentrator 100 is to provide supplemental oxygen to a user. One or more flow rate settings may be selected on a control panel 600 of the oxygen concentrator 100, which then will control operations to achieve production of the oxygen enriched air according to the selected flow rate setting. In some versions, a plurality of flow rate settings may be implemented (e.g., five flow rate settings). As described in more detail herein, the controller may implement a POD (pulsed oxygen delivery) or demand mode of operation to regulate size of one or more released boluses to achieve delivery of the oxygen enriched air according to the selected flow rate setting.

In order to maximise the effect of the delivered oxygen enriched air, controller 400 may be programmed to synchronise release of each bolus of the oxygen enriched air with the user's inhalations. Releasing a bolus of oxygen enriched air to the user as the user inhales may prevent wastage of oxygen by not releasing oxygen, for example, when the user is exhaling. For concentrators that operate in POD mode, the flow rate settings on the control panel 600 may correspond to minute volumes (bolus volume multiplied by breathing rate per minute) of delivered oxygen, e.g. 0.2 LPM, 0.4 LPM, 0.6 LPM, 0.8 LPM, 1.1 LPM.

Oxygen enriched air produced by oxygen concentrator 100 is stored in an oxygen accumulator 106 and, in POD mode, released to the user as the user inhales. The amount of oxygen enriched air provided by the oxygen concentrator 100 is controlled, in part, by supply valve 160. In an implementation, supply valve 160 is actuated (opened) for a sufficient amount of time to provide the appropriate amount of oxygen enriched air, as estimated by controller 400, to the user. In order to minimize the wastage of oxygen, the oxygen enriched air may be provided as a bolus soon after the onset of a user's inhalation is detected. For example, the bolus of oxygen enriched air may be provided in the first few milliseconds of a user's inhalation.

In an implementation, pressure sensor 194 may be used to determine the onset of inhalation by the user. For example, the user's inhalation may be detected by using pressure sensor 194. In use, conduit 192 for providing oxygen enriched air is coupled to a user's nose and/or mouth through the nasal airway delivery device 196 and/or mouthpiece 198. The pressure in conduit 192 is therefore representative of the user's airway pressure and therefore indicative of user respiration. At the onset of an inhalation, the user begins to draw air into their body through the nose and/or mouth. As the air is drawn in, a negative pressure is generated at the end of the conduit 192, due, in part, to the venturi action of the air being drawn across the end of the conduit. Controller 400 analyses the pressure signal from the pressure sensor 194 to detect a drop in pressure indicating the onset of inhalation. Upon detection of the onset of inhalation, supply valve 160 is opened to release a bolus of oxygen enriched air from the accumulator 106. A positive change or rise in the pressure indicates an exhalation by the user, upon which the release of oxygen enriched air is discontinued. In one implementation, when a positive pressure change is sensed, supply valve 160 is closed until the next onset of inhalation is detected. Alternatively, supply valve 160 may be closed after a predetermined interval known as the bolus duration. By measuring the intervals between adjacent onsets of inhalation, the user's breathing rate may be estimated. By measuring the intervals between onsets of inhalation and the subsequent onsets of exhalation, the user's inspiratory time may be estimated. Thus, the user's breathing rate or respiration rate may be detected with a signal from the pressure sensor and/or a flow rate sensor.

In other implementations, the pressure sensor 194 may be located in a sensing conduit that is in pneumatic communication with the user's airway, but separate from the delivery conduit 192. In such implementations the pressure signal from the pressure sensor 194 is therefore also representative of the user's airway pressure.

In some implementations, the sensitivity of the pressure sensor 194 may be affected by the physical distance of the pressure sensor 194 from the user, especially if the pressure sensor 194 is located in oxygen concentrator 100 and the pressure difference is detected through the conduit 192 coupling the oxygen concentrator 100 to the user. In some implementations, the pressure sensor 194 may be placed in the airway delivery device 196 used to provide the oxygen enriched air to the user. A signal from the pressure sensor 194 may be provided to controller 400 in the oxygen concentrator 100 electronically via a wire or through telemetry such as through Bluetooth™ or other wireless technology.

In some implementations, if the user's current activity level, such as that estimated using the detected user's breathing rate, exceeds a predetermined threshold, controller 400 may implement an alarm (e.g., visual and/or audio) to warn the user that the current breathing rate is exceeding the delivery capacity of the oxygen concentrator 100. For example, the threshold may be set at 40 breaths per minute (BPM).

### Bolus Size Regulation

As previously described, an oxygen concentrator may employ a conserver or the controller may implement a conserver such as by controlling oxygen enriched air release in a pulsed or demand therapy mode. This may be achieved by delivering the oxygen as a series of pulses, where each pulse or “bolus” may be timed to coincide with inspiration. Such a mode is typically controlled by actuating the supply valve 160 for a fixed time, where the fixed time starts with the opening of the supply valve 160 to permit release of the bolus and ends when the supply valve 160 is closed thereby to stop releasing the bolus. The fixed time is calibrated to be associated with a desired or target bolus size, e.g. a target bolus volume. However, such a fixed-time process does not always achieve the target bolus volume. For example, system characteristics such as compressor variability as well as the adsorption process (e.g., the PSA cycle, sieve bed condition, air filter condition etc.) can interfere with the delivered bolus volume. Accordingly, examples of the present technology may provide improved control of the bolus release in a POD mode to permit greater consistency and/or accuracy of released bolus size. As discussed in more detail herein, bolus release control may be implemented with a dynamic timing parameter (e.g., a timing threshold), rather than a fixed time, that may take into account changing system conditions during release of the bolus so that the bolus control may more accurately achieve the desired size. Thus, the timing threshold for stopping the bolus release may change during the release of the bolus depending on system conditions (e.g., pressure). Examples of such technology may be understood in more detail in relation to FIGS. 15 to 17 and 19. For example, the release of a bolus may be implemented by the controller applying a function of a value of a measured pressure signal from a pressure sensor, such as an accumulator pressure sensor, to obtain a target duration for valve opening for the bolus. The controller may dynamically determine, calculate (or recalculate) the target duration during the release of the bolus, such as with the function. Moreover, the function may include one or more parameters (e.g., empirical constants) of a model pressure-time-volume surface that is derived from a calibration process utilizing measured pressure, bolus volume and valve actuation times. Parameters for such a modelled surface may optionally be derived by a best fitting process such as regression utilizing the calibration measurements.

An example of such a dynamically controlled bolus release may be considered in relation to the flow chart of FIG. 15, which illustrates a method 1500 that may be implemented by a controller 400 of the POC 100. At 1502, the controller may evaluate a signal indicative of user respiration (e.g., detect the onset of inhalation) from a sensor, e.g. pressure sensor 194, configured to generate a signal indicative of respiration, to detect an inspiration characteristic. As previously described, this may involve detecting a drop in pressure associated with the onset of user inhalation. Responsive to the signal indicative of respiration (e.g. based on such a detection of a drop in pressure), the controller, at 1504, may open the supply valve 160 to initiate release of a bolus. At 1506, the controller may then monitor one or more system characteristics during the bolus release, such as a measure of pressure of accumulated oxygen enriched air. For example, the controller may determine or update an average accumulator pressure such as by summing samples from a signal from the accumulator pressure sensor 107 where the average particularly concerns the period of the bolus release, that is, when the supply valve is open for the bolus release. At 1508, the controller may then calculate (or recalculate) a timing threshold (e.g., a target duration) for actuation of the supply valve 160 so as to determine when it may be closed. The timing threshold may be determined or calculated for achieving a target or desired bolus size and may be based on measured pressure (e.g., average pressure) and/or a modelled surface based on volume, pressure and valve opening times, such as using a function described in more detail herein. At 1510, the controller may compare the calculated timing threshold to an elapsed time, such as from a timer, that corresponds to an amount of time that the supply valve 160 has been open for releasing the bolus (e.g., elapsed time >target duration and/or elapsed time=target duration). Based on the comparison (e.g., “YES”), the controller may proceed to close the supply valve to stop the bolus release at 1512, such as if the target duration has been achieved. Alternatively, based on the comparison (e.g., “NO”), the controller may return to monitoring of the system characteristics for updating the average pressure with additional samples from accumulator pressure sensor while the supply valve 160 remains open such that the process repeats 1506, 1508 and 1510. This repetition permits the dynamic adjustment of the timing threshold. With the closing of the supply valve at 1512, a bolus has been released according to the desired volume and the process 1500 may return to 1502.

In some implementations the rate of iteration through the loop formed by the steps 1506, 1508, and 1510 is sufficiently fast that the resulting resolution of supply valve opening time is adequate to allow the bolus size to reliably approximate the target size. For example, the rate of iteration may be 1000 Hertz (Hz), giving the supply valve opening time a resolution of ±0.5 milliseconds (ms). For a typical supply valve opening time of 150 milliseconds (ms), this represents an accuracy of less than 1%. In some implementations not every iteration of step 1506 provides an updated measure of pressure. In such implementations the previous measure of pressure may be used at iterations of step 1506 where the measure of pressure has not been updated.

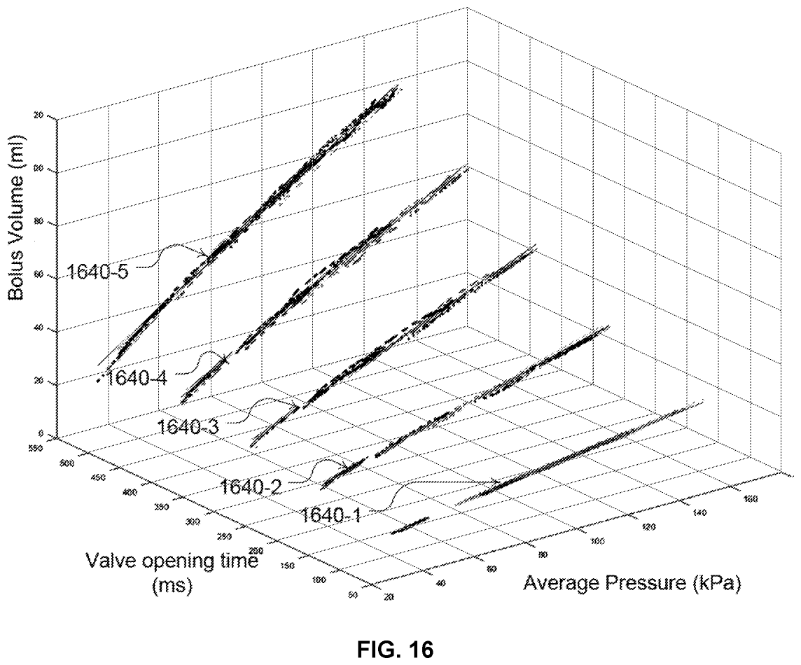

Accordingly, in some of these examples as previously discussed, the controller may implement a function for controlling release of the bolus so that the bolus is regulated to achieve a desired bolus size, e.g. volume. The function may comprise a modelled surface using modelling coefficients. The modelled surface may be empirically derived such as in a calibration process. The function may be derived to map pressure (e.g., average pressure) and valve opening times to bolus size, such as for one or more flow settings of the portable oxygen concentrator 100, and may include one or more modelling coefficients (empirical constants). An example function that is suitable for some versions of the present technology may be considered in relation to FIG. 16. FIG. 16 plots points representing empirically determined values relating average pressure during bolus delivery and supply valve opening times to delivered bolus volumes. As shown in the graph, each set of points 1640-1, 1640-2, 1640-3, 1640-4, 1640-5 may be determined in relation to different supply valve opening times of the portable oxygen concentrator 100. With measured values (shown as points in FIG. 16) of bolus volume delivered by the POC, a modelled surface may be derived from or fitted to the data, such as by regression or best fit analysis, to derive the parameters (e.g., the modelling coefficients or constants) of the surface. In the examples utilizing pressure and valve opening times for a range of bolus volumes, a suitable function for the data may be as follows:

BolusSize=a*P+b*P*Ftime+c*Ftime+d EQ. 1

where:

BolusSize is a volume for the bolus such as in milliliters; P is a value of measured pressure, such as an average pressure during bolus release, or a pressure measure at the time of initial bolus release;

Fₜᵢₘₑ is a duration or period of time that the supply valve is open during release of the bolus; and

a, b, c, and d are empirical constants derived from a surface fitting process applied to the calibration process measurements illustrated in FIG. 16. Thus, the modelled surface may be bilinear or another suitable shape. The calibration may be conducted for each POC 100 individually, in which case the empirical constants may be different for each POC, or for a single POC that is representative of multiple POCs with similar outlet pneumatic characteristics, in which case the multiple POCs may share a common set of empirical constants.

The derived function may then be programmed into a controller for the regulation of bolus size in a POD mode. For example, as discussed in relation to the examples herein, the function may be applied for dynamic determination or calculation of the timing threshold (e.g., a target duration) for delivery of a bolus of a desired size by inverting the equation of EQ. 1 to obtain the following function:

$$TargetDuration = \frac{( TargetBolusSize - a * P - d )}{( b * P + c )} \qquad EQ . 2$$

where:
TargetDuration is the target duration that may be implemented as a timing threshold for bolus delivery (i.e., the supply valve opening time from the start of the bolus to the end of the bolus or the elapsed time of the release of the bolus);
TargetBolusSize is a target bolus volume, such as a target volume associated with a breathing rate of a user and a flow setting of the POC;
P is a value of measured pressure during bolus release corresponding to the values of measured pressure used during calibration, such as an average pressure during bolus release; and
a, b, c and d are the empirical constants from the modelled surface. In some implementations, a determined set of these constants may be associated with each flow setting of the oxygen concentrator. Thus, when the function is applied by a controller in a POD mode, the controller may access a particular set of constants that is associated with a currently applied flow setting of the POC. Thus, the controller may have a plurality of discrete sets of empirical constants for the surface that are respectively associated with different flow settings of the oxygen concentrator. In some such implementations, each discrete set of empirical constants is associated with a group of flow settings, e.g. flow settings 1 to 3. Such an associated group may consist of one or more flow settings. In other implementations, a single set of empirical constants may be derived and applied for all flow settings.

In alternative implementations, the function may be based on other modelled surfaces with different parameters to be fitted to the data acquired during calibration. One such alternative implementation uses a second-order modelled surface defined as:

BolusSize=a*P²*Ftime+b*P²+c*P*Ftime+d*Ftime EQ. 3

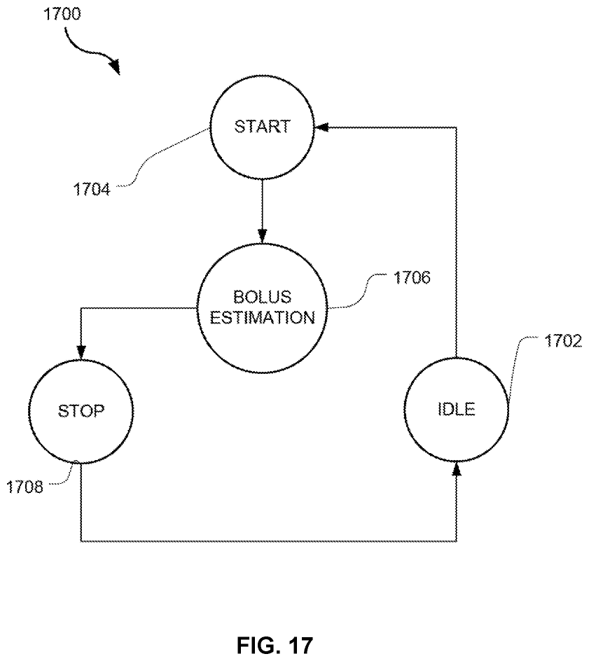

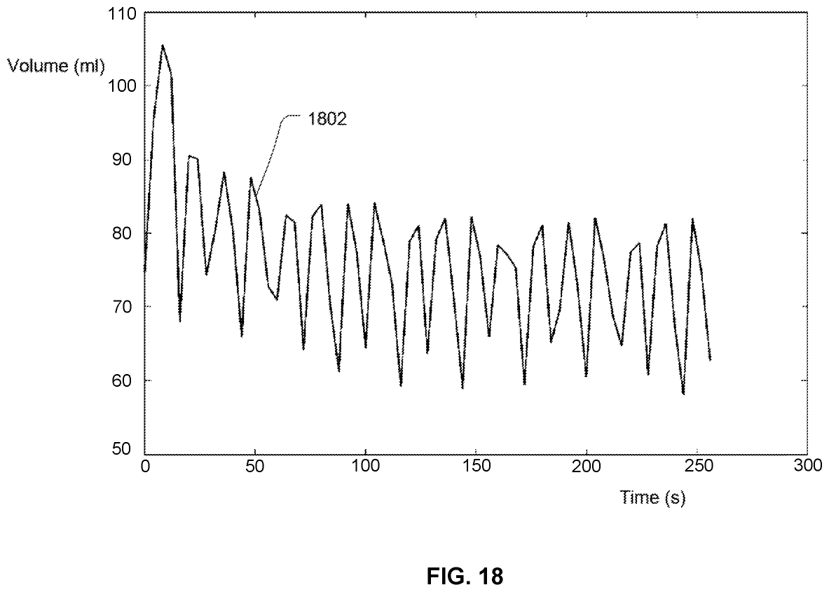

As previously described, such a function may be implemented in a POC controller (e.g., controller 400) to dynamically adjust bolus release duration for achieving a desired bolus size. For example, a controller of a POC may be configured to implement a state machine for implementing a POD mode of operation for dynamically regulating release of one or more boluses to a desired volume. An example of such a state machine 1700 may be considered in relation to the state diagram of FIG. 17. A controller, to regulate bolus size (e.g., volume), may be configured with any one, more or all of an idle state 1702, a start state 1704, a bolus estimation state 1706 and a stop state 1708. In the idle state 1702, the controller 400 may be configured to detect or evaluate a characteristic of user inspiration, such as from a signal indicative of patient respiration. Optionally, the controller, in the idle state 1702 may await a trigger signal for starting bolus release.

The controller may transition to the start state 1704, from the idle state 1702 on detection of the condition for activating a bolus release as previously discussed. In the state machine 1700, the controller may operate a timer to determine elapsed time since the opening of the supply valve. For this purpose, the controller may initialize the timer when transitioning into the start state 1704. Optionally, in the case of use of a value of the pressure signal such as an average pressure, the controller may initialize the value of the average pressure, such as to zero. In the start state 1704, the controller may also determine or calculate a target bolus size. For example, the target bolus size may be determined as a function of a present flow setting of the POC (e.g., a minute volume) and a current breathing rate of the user. As previously described, such a breathing rate may be determined with a respiration sensor such as the pressure sensor 194. In the example, the target bolus size may be determined by dividing the minute volume by the breathing rate (e.g., in breaths per minute (BPM)).

In some implementations, the minute volume Vₘ associated with the present flow setting may be adjusted depending on the current breathing rate. In one such implementation, the minute volume Vₘ may be adjusted in proportion to the amount by which the current breathing rate departs from a reference breathing rate (e.g. 20 BPM). The target bolus size may then be determined by dividing the adjusted minute volume by the current breathing rate. In one such implementation, the adjusted minute volume [symbol] may be computed as

[symbol]

=Vₘ−k(BPM−BPM_{ref}) (1)

where BPM_{ref} is a reference breathing rate and k is a constant of proportionality. For example, if k is positive the effect of this adjustment is to reduce the target bolus size when the patient is breathing at a rate faster than the reference breathing rate.

The controller 400 may then, in the start state 1704, generate a signal to open the supply valve 160. The timer may then begin to be incremented for establishing elapsed time starting with this signal generation. The controller may then begin summing samples of measured pressure, such as from the accumulator pressure sensor 107, for determining an average of the summed samples. Thus, the controller may maintain a cumulative sum and a total number of samples for calculating the average. These maintaining operations of the controller in the start state 1704 may continue for a predetermined minimum period of time. In this regard, the controller may transition from the start state 1704 to the bolus estimation state 1706 once the elapsed time from the opening of the supply valve 160 for the bolus exceeds the minimum period of time such as by a comparison of the elapsed time of the timer and a threshold representing the minimum period of time. Thus, at the transition from the start state 1704 to the bolus estimation state 1706, the calculation of average pressure may have been ongoing for a minimum period of time and the supply valve may have been open for that minimum period of time. In other words, the controller at least refrains from closing the supply valve until the elapsed time meets or exceeds the minimum period of time.

In the bolus estimation state 1706, the controller maintains the supply valve 160 in the open state. Moreover, the controller may continue to use samples of the measured pressure to repeatedly update the average pressure while in the bolus estimation state. To this end, pressure samples may be repeatedly summed with the cumulative sum, which is then divided by the total number of samples. The controller may then dynamically determine or calculate a target duration (a timing threshold) such by using a function (e.g., EQ. 2) of a value of measured pressure, such as the average pressure determined while the supply valve is open, for release of the bolus (e.g., from the start state 1704 and the bolus estimation state 1706 combined) so as to ensure that the appropriate bolus size is released. The controller may then compare the target duration with the elapsed time of the timer initialized in the start state 1704. The controller may transition to the stop state 1708 when the elapsed time equals or exceeds the dynamically determined target duration, thereby ensuring release of the desired bolus size. In this regard, the controller, in the bolus estimation state 1706, may repeatedly update the average pressure and the target duration for repeating the comparison until transitioning to the stop state 1708 when the target duration is reached. Optionally, the controller may also transition to the stop state 1708 if the elapsed time exceeds a maximum time. In the stop state 1708, the controller closes the supply valve 160 to stop the bolus release, such as by discontinuing the signal for opening the supply valve 160. The controller may then transition to the idle state 1702 to await the next cycle for bolus release.

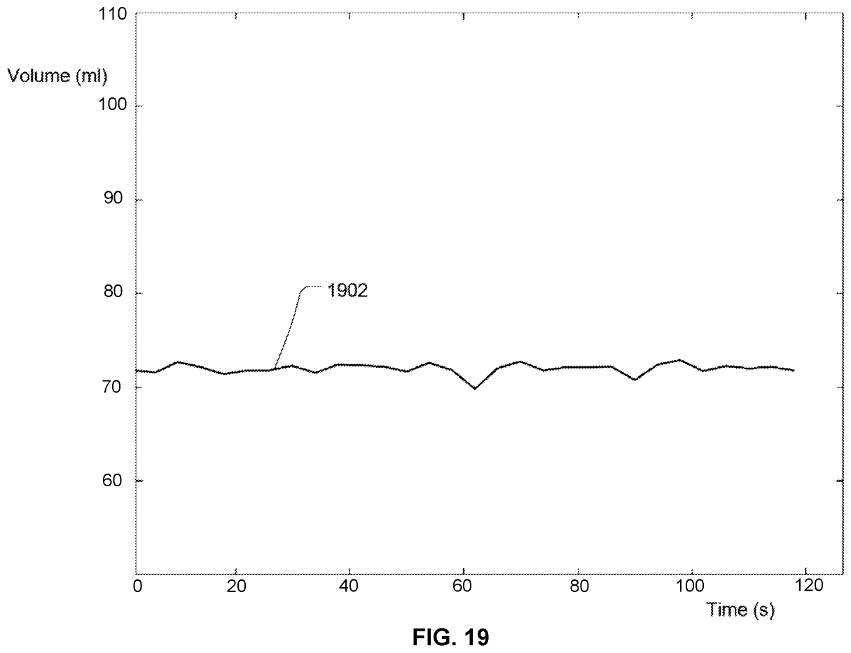

These example processes of the controller for regulating bolus size can improve consistency of the oxygen therapy over time. Such consistency may be considered in relation to the graphs of FIGS. 18 and 19. These figures each illustrate a trace of bolus size (volume in this case) over time. FIG. 18 illustrates operation of a POC utilizing a fixed timing threshold. As shown, the variation of the trace 1802 over time illustrates that the POC with such valve control can produce inconsistent bolus sizes, which may be a result of variations in system characteristics. FIG. 19 illustrates operation of a POC utilizing the dynamically determined target duration for control of the supply valve as described in the aforementioned examples such as using average pressure in regulating bolus size. As seen in the graph of FIG. 19, which has the same vertical scale as FIG. 18, the variation of the trace 1902 over time illustrates that the POC with such valve control can produce greater consistency in bolus size despite variations in system characteristics.

In some implementations, the valve opening time computed according to the state machine 1700 may be implemented to compensate for one or more effects that may perturb the delivered bolus size away from the desired bolus size. One such effect is temperature. If the temperature of the oxygen-enriched gas is significantly different from the temperature that prevailed during the calibration that produced the empirical constants of the modelled pressure-time-volume surface, the modelled surface will be a less accurate predictor of bolus size for a given average pressure and supply valve opening time.

To compensate for system temperature, a gas temperature in the outlet system (e.g. inside the accumulator 106) may be measured by a temperature sensor as previously mentioned. A function may be applied to the measured temperature and the target bolus size (e.g. during the start state 1704 of the state machine 1700) to produce a temperature-adjusted target bolus size. In one implementation of such temperature adjustment, the target bolus volume may be changed in proportion to the change in absolute temperature relative to the temperature existing when the calibration was performed. For example, the target bolus volume may be increased in proportion to the increase in absolute temperature since the calibration was performed. The timing threshold may then be calculated by applying the function using the temperature-adjusted target bolus size at step 1508.

### Glossary

For the purposes of the present technology disclosure, in certain forms of the present technology, one or more of the following definitions may apply. In other forms of the present technology, alternative definitions may apply.

Air: In certain forms of the present technology, air may be taken to mean atmospheric air, consisting of 78% nitrogen (N₂), 21% oxygen (O₂), and 1% water vapour, carbon dioxide (CO₂), argon (Ar), and other trace gases.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Ambient: In certain forms of the present technology, the term ambient will be taken to mean (i) external of the treatment system or user, and (ii) immediately surrounding the treatment system or user.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

Patient: A person, whether or not they are suffering from a respiratory disorder.

Pressure: Force per unit area. Pressure may be expressed in a range of units, including cmH₂O, g-f/cm2 and hectopascal. 1 cmH₂O is equal to 1 g-f/cm2 and is approximately 0.98 hectopascal (1 hectopascal=100 Pa=100 N/m²=1 millibar˜0.001 atm). In this specification, unless otherwise stated, pressure is given in units of cmH₂O.

### GENERAL REMARKS

The term “coupled” as used herein means either a direct connection or an indirect connection (e.g., one or more intervening connections) between one or more objects or components. The phrase “connected” means a direct connection between objects or components such that the objects or components are connected directly to each other. As used herein the phrase “obtaining” a device means that the device is either purchased or constructed.

In the present disclosure, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative implementations of various aspects of the present technology may be apparent to those skilled in the art in view of this description. Accordingly, this description is to be construed as illustrative only and is for the purpose of teaching those skilled in the art the general manner of carrying out the technology. It is to be understood that the forms of the technology shown and described herein are to be taken as implementations. Elements and materials may be substituted for those illustrated and described herein, parts and processes may be reversed, and certain features of the technology may be utilized independently, all as would be apparent to one skilled in the art after having the benefit of this description of the technology. Changes may be made in the elements described herein without departing from the spirit and scope of the technology as described in the appended claims.

### LABEL LIST

oxygen concentrator 100
cooling system passive inlet 101
air inlet 105
accumulator 106
accumulator pressure sensor 107
muffler 108
valve 122
valve 124
filter 129
outlet 130
valve 132
muffler 133
valve 134
baffle 139
check valve 142
check valve 144
flow restrictor 151
valve 152
flow restrictor 153
valve 154
flow restrictor 155
supply valve 160
expansion chamber 162
oxygen sensor 165
emitters 166
ultrasonic receiver 168
outer housing 170
fan 172
outlet 173
outlet port 174
flow restrictor 175
power supply 180
flow rate sensor 185
particulate filter 187
connector 190
conduit 192
pressure sensor 194
nasal airway delivery device 196
mouthpiece 198
compression system 200
speed sensor 201
compressor 210
compressor outlet 212
motor 220
external armature 230
air transfer device 240
compressor outlet conduit 250
canister system 300
canister 302
canister 304
air inlet 306
housing 310
base 315
valve seats 322
openings 323
valve seats 324
outlet 325
gases 327
conduit 330
valve seat 332
apertures 337
conduits 342
conduit 344
conduit 346
opening 375
controller 400
processor 410
memory 420
transceiver 430
housing component 510
conduit 530
conduit 532
conduit 534
links openings 542
opening 544
valve seat 552
valve seat 554
control panel 600
input port 605
power button 610
button 620
button 622
button 624
button 626
button 630
button 635
altitude button 640
battery check button 650
LED 655
method 1500
step 1502
step 1504
step 1506
step 1508
step 1510
step 1512
points 1640-1
points 1640-2
points 1640-3
points 1640-4
points 1640-5
state machine 1700
idle state 1702
start state 1704
bolus estimation state 1706
stop state 1708
trace 1802
trace 1902

1. A method of operating an oxygen concentrator, the method comprising:
generating, with a sensor configured to sense pressure at a location associated with accumulation of oxygen enriched air produced by the oxygen concentrator, a signal representing measured pressure of the accumulated oxygen enriched air;
generating, with a sensor, a signal indicative of respiration of a user of the oxygen concentrator; and
with a controller configured to receive the signal representing measured pressure and the signal indicative of a respiration of the user, controlling, responsive to the signal indicative of respiration and according to a target duration, actuation of a valve adapted to release a bolus of the accumulated oxygen enriched air,
wherein the controller dynamically determines the target duration during the release of the bolus according to a function of a value of the measured pressure.

2. The method of claim 1 wherein the controller controls actuation of the valve by (a) opening the valve to initiate release of the bolus at a first time associated with a detection of an inspiration characteristic in the signal indicative of respiration of the user, and (b) closing the valve when elapsed time from the first time meets or exceeds the target duration.

3. The method of claim 2 wherein the controller closes the valve when the elapsed time from the first time meets or exceeds a maximum time.

4. The method of any one of claims 2 to 3 wherein the controller refrains from closing the valve until the elapsed time from the first time meets or exceeds a minimum time.

5. The method of any one of claims 2 to 4 wherein the value of the measured pressure is a calculated average.

6. The method of claim 5 wherein the calculated average is an average pressure during the release of the bolus.

7. The method of claim 6 wherein the controller (a) repeatedly updates the average pressure and the target duration during the release of the bolus, and (b) repeatedly compares the elapsed time with the updated target duration during the release of the bolus.

8. The method of any one of claims 1 to 7 wherein the function comprises a target bolus size.

9. The method of claim 8 wherein the controller calculates the target bolus size as a function of a detected respiration rate of the user and a flow rate associated with a flow setting of the oxygen concentrator.

10. The method of any one of claims 8 to 9, further comprising generating, with a sensor, a signal indicative of a temperature of the accumulated oxygen enriched air.

11. The method of claim 10, wherein the controller adjusts the target bolus size dependent on the signal indicative of the temperature of the accumulated oxygen enriched air.

12. The method of any one of claims 1 to 11 wherein the function comprises a plurality of empirical constants of a modelled surface derived from pressure values and valve opening times of a calibration process.

13. The method of claim 12 wherein the modelled surface is bilinear.

14. The method of claim 13 wherein the function comprises:
$$TargetDuration = \frac{( TargetBolusSize - a * P - d )}{( b * P + c )}$$
where:
TargetDuration is the target duration;
TargetBolusSize is a target bolus size;
P is the value of the measured pressure; and
a, b, c and d are the empirical constants.

15. The method of any one of claims 12 to 14 wherein the empirical constants comprise a selected set of empirical constants associated with a flow rate setting of the oxygen concentrator, the selected set being chosen from a plurality of discrete sets of empirical constants that are respectively associated with a plurality of discrete flow rate settings of the oxygen concentrator.

16. The method of any one of claims 1 to 15 wherein the controller comprises: an idle state, a start state, a bolus estimation state, and a stop state.

17. The method of claim 16 wherein the controller transitions from the idle state to the start state upon detection of an inspiration characteristic in the signal indicative of respiration of the user.

18. The method of claim 17 wherein the controller, in the start state, generates a signal to open the valve, and initializes a valve timer.

19. The method of claim 18 wherein the controller, in the start state, calculates an average pressure value with samples taken from the signal representing measured pressure in the start state.

20. The method of claim 19 wherein the controller transitions to the bolus estimation state from the start state when the valve timer exceeds a minimum time.

21. The method of claim 20 wherein the controller, in the bolus estimation state, repeatedly calculates a target duration with the average pressure value.

22. The method of any one of claims 20 to 21 wherein the controller, in the bolus estimation state, repeatedly calculates the average pressure value with samples taken from the signal representing measured pressure in the bolus estimation state.

23. The method of any one of claims 20 to 22 wherein the controller, in the bolus estimation state, repeatedly compares the target duration with the valve timer.

24. The method of claim 23 wherein the controller transitions to the stop state when (a) the valve timer meets or exceeds the target duration, or (b) when the valve timer meets or exceeds a maximum time.

25. The method of any one of claims 18 to 24 wherein the controller, in the stop state, stops generating the signal to open the valve.

26. An oxygen concentrator comprising:
one or more sieve beds containing a gas separation adsorbent;
a compression system, including a motor operated compressor, configured to feed a feed gas into the one or more sieve beds;
an accumulator configured to receive oxygen enriched air from the one or more sieve beds;
a respiration sensor configured to generate a signal indicative of respiration of a user of the oxygen concentrator;
a pressure sensor configured to generate a signal representing measured pressure of the oxygen enriched air in the accumulator;
a valve adapted to release a bolus of the oxygen enriched air from the accumulator;
a memory; and
a controller comprising one or more processors, the one or more processors configured by program instructions stored in the memory to execute the method of operating the oxygen concentrator according to the method of any one of claims 1 to 25.

27. A computer-readable medium having encoded thereon computer-readable instructions that when executed by a controller of an oxygen concentrator cause the controller to perform the method of operating the oxygen concentrator of any one of claims 1 to 25.

28. An oxygen concentrator comprising:
one or more sieve beds containing a gas separation adsorbent;
a compression system, including a motor operated compressor, configured to feed a feed gas into the one or more sieve beds;
an accumulator to receive oxygen enriched air from the one or more sieve beds;
a pressure sensor configured to generate a signal representing measured pressure of the oxygen enriched air in the accumulator;
a respiration sensor configured to generate a signal indicative of respiration of a user of the oxygen concentrator;
a valve adapted to release a bolus of the oxygen enriched air from the accumulator; and
a controller coupled with the pressure sensor, the respiration sensor and the valve, the controller configured to:
receive the signal representing measured pressure;
receive the signal indicative of respiration; and
control, responsive to the signal indicative of respiration and according to a target duration, actuation of the valve to release the bolus of the oxygen enriched air, wherein the controller is configured to dynamically determine the target duration during the release of the bolus according to a function of a value of the measured pressure.

29. The oxygen concentrator of claim 28 wherein the controller is configured to control actuation of the valve by (a) opening the valve to initiate release of the bolus at a first time associated with a detection of an inspiration characteristic in the signal indicative of respiration of the user, and (b) closing the valve when elapsed time from the first time meets or exceeds the target duration.

30. The oxygen concentrator of claim 29 wherein the controller is configured to close the valve when the elapsed time from the first time meets or exceeds a maximum time.

31. The oxygen concentrator of any one of claims 29 to 30 wherein the controller is configured to refrain from closing the valve until the elapsed time from the first time meets or exceeds a minimum time.

32. The oxygen concentrator of any one of claims 29 to 31 wherein the value of the measured pressure is a calculated average.

33. The oxygen concentrator of claim 32 wherein the calculated average is an average pressure during the release of the bolus.

34. The oxygen concentrator of claim 33 wherein the controller is configured to (a) repeatedly update the average pressure and the target duration during the release of the bolus, and (b) repeatedly compare the elapsed time with the updated target duration during the release of the bolus.

35. The oxygen concentrator of any one of claims 28 to 34 wherein the function comprises a target bolus size.

36. The oxygen concentrator of claim 35 wherein the controller is configured to calculate the target bolus size as a function of a detected respiration rate of the user and a flow rate associated with a flow setting of the oxygen concentrator.

37. The oxygen concentrator of any one of claims 35 to 36 further comprising a sensor configured to generate a signal indicative of a temperature of the oxygen enriched air in the accumulator.

38. The oxygen concentrator of claim 37 wherein the controller is configured to adjust the target bolus size dependent on the signal indicative of the temperature of the oxygen enriched air.

39. The oxygen concentrator of any one of claims 28 to 38 wherein the function comprises a plurality of empirical constants of a modelled surface derived from pressure values and valve opening times of a calibration process.

40. The oxygen concentrator of claim 39 wherein the modelled surface is bilinear.

41. The oxygen concentrator of claim 40 wherein the function comprises:
$$TargetDuration = \frac{( TargetBolusSize - a * P - d )}{( b * P + c )}$$
where:
TargetDuration is the target duration;
TargetBolusSize is a target bolus size;
P is the value of the measured pressure; and
a, b, c and d are the empirical constants.

42. The oxygen concentrator of any one of claims 39 to 41 wherein the empirical constants comprise a selected set of empirical constants associated with a flow rate setting of the oxygen concentrator, the controller is configured to choose the selected set from a plurality of discrete sets of empirical constants that are respectively associated with a plurality of discrete flow rate settings of the oxygen concentrator.

43. The oxygen concentrator of any one of claims 28 to 42 wherein to regulate bolus release, the controller is configured with: an idle state, a start state, a bolus estimation state and a stop state.

44. The oxygen concentrator of claim 43 wherein the controller is configured to transition from the idle state to the start state upon detection of an inspiration characteristic in the signal indicative of respiration of the user.

45. The oxygen concentrator of claim 44 wherein the controller, in the start state, is configured to generate a signal to open the valve, and initialize a valve timer.

46. The oxygen concentrator of claim 45 wherein the controller, in the start state, is configured to calculate an average pressure value with samples taken from the signal representing measured pressure in the start state.

47. The oxygen concentrator of claim 46 wherein the controller is configured to transition to the bolus estimation state from the start state when the valve timer exceeds a minimum time.

48. The oxygen concentrator of claim 47 wherein the controller, in the bolus estimation state, is configured to repeatedly calculate a target duration with the average pressure value.

49. The oxygen concentrator of any one of claims 47 to 48 wherein the controller, in the bolus estimation state, is configured to repeatedly calculate the average pressure value with samples taken from the signal representing measured pressure in the bolus estimation state.

50. The oxygen concentrator of claim 49 wherein the controller, in the bolus estimation state, is configured to repeatedly compare the target duration with the valve timer.

51. The oxygen concentrator of claim 50 wherein the controller is configured to transition to the stop state when (a) the valve timer meets or exceeds the target duration, or (b) when the valve timer meets or exceeds a maximum time.

52. The oxygen concentrator of any one of claims 45 to 51 wherein the controller, in the stop state, is configured to stop generating the signal to open the valve.

53. Apparatus comprising
bed means for containing a gas separation adsorbent;
means for feeding a feed gas into the bed means;
accumulation means for receiving oxygen enriched air from the bed means;
pressure sensing means for generating a signal representing measured pressure of the oxygen enriched air in the accumulation means;
respiration sensing means for generating a signal indicative of respiration of a user of the apparatus;
releasing means adapted to release a bolus of the oxygen enriched air from the accumulation means; and
controlling means coupled with the pressure sensing means, the respiration sensing means and the releasing means, the controlling means for:
receiving the signal representing measured pressure;
receiving the signal indicative of respiration;
controlling, responsive to the signal indicative of respiration and according to a target duration, actuation of the releasing means to release the bolus of the oxygen enriched air; and
dynamically determining the target duration during the release of the bolus according to a function of a value of the measured pressure.
